SEQUENCE LISTING (54 sequences)

SEQ ID NO: 1 - protein, 32 aa, herpes simplex virus 7
NKSLGACPIRTQPRWNYYDSFSAVSEDNLGFL

SEQ ID NO: 2 - protein, 34 aa, herpes simplex virus 7
QPPSLPITVYYAVLERACRSVLLNAPSEAPQIVR

SEQ ID NO: 3 - protein, 31 aa, herpes simplex virus 7
ITQFILEHRAKGSCKYALPLRIPPSACLSPQ

SEQ ID NO: 4 - protein, 35 aa, herpes simplex virus 7
SACLSPQAYQQGVTVDSIGMLPRFIPENQRTVAVY

SEQ ID NO: 5 - protein, 28 aa, herpes simplex virus 7
TIAWFRMGGNCAIPITVMEYTECSYNKS

SEQ ID NO: 6 - protein, 28 aa, herpes simplex virus 7
APQIVRGASEDVRKQPYNLTIAWFRMGG

SEQ ID NO: 7 - protein, 34 aa, herpes simplex virus 7
EDNLGFLMHAPAFETAGTYLRLVKINDWTEITQF

SEQ ID NO: 8 - protein, 30 aa, herpes simplex virus 7
QRTVAVYSLKIAGWHGPKAPYTSTLLPPEL

SEQ ID NO: 9 - protein, 32 aa, herpes simplex virus 7
DLPVLDQLTDPPGVRRVYHIQAGLPDPFQPPS

SEQ ID NO: 10 - protein, 26 aa, herpes simplex virus 7
ICGVYWMRRHTQKAPKRIRLPHIRED

SEQ ID NO: 11 - protein, 29 aa, herpes simplex virus 7
SKYALVDASLKMADPNRFRGKDLPVLDQL

SEQ ID NO: 12 - protein, 23 aa, herpes simplex virus 7
KYALVDASLKMADPNRFRGKDLP

SEQ ID NO: 13 - protein, 31 aa, herpes simplex virus 7
APQIPPNWHIPSIQDAATPYHPPATPNNMGL

SEQ ID NO: 14 - protein, 35 aa, herpes simplex virus 7
FRYVMRLQSNPMKALYPLTTKELKNPTNPDASGEG

SEQ ID NO: 15 - protein, 40 aa, herpes simplex virus 7
VEEVDARSVYPYDEFVLATGDFVYMSPFYGYREGSHTEHT

SEQ ID NO: 16 - protein, 30 aa, herpes simplex virus 7
NYTEGIAVVFKENIAPYKFKATMYYKDVTV

SEQ ID NO: 17 - protein, 32 aa, herpes simplex virus 7
KLAEAREMIRYMALVSAMERTEHKAKKKGTSA

SEQ ID NO: 18 - protein, 33 aa, herpes simplex virus 7
ATHIKVGQPQYYLANGGFLIAYQPLLSNTLAEL

SEQ ID NO: 19 - protein, 28 aa, herpes simplex virus 7
HRRYFTFGGGYVYFEEYAYSHQLSRADI

SEQ ID NO: 20 - protein, 31 aa, herpes simplex virus 7
RADITTVSTFIDLNITMLEDHEFVPLEVYTR

SEQ ID NO: 21 - protein, 23 aa, herpes simplex virus 7
NNELRLTRDAIEPCTVGHRRYFT

SEQ ID NO: 22 - protein, 22 aa, herpes simplex virus 7
PLLSNTLAELYVREHLREQSRK

SEQ ID NO: 23 - protein, 32 aa, herpes simplex virus 7
AKGVCRSTAKYVRNNLETTAFHRDDHETDMEL

SEQ ID NO: 24 - protein, 36 aa, herpes simplex virus 7
PPGASANASVERIKTTSSIEFARLQFARLQFTYNHI

SEQ ID NO: 25 - protein, 27 aa, herpes simplex virus 7
DANFYVCPPPTGATVVQFEQPRRCPTR

SEQ ID NO: 26 - protein, 34 aa, herpes simplex virus 7
GTSALLSAKVTDMVMRKRRNTNYTQVPNKDGDAD

SEQ ID NO: 27 - protein, 27 aa, herpes simplex virus 7
SRPLVSFRYEDQGPLVEGQLGENNELR

SEQ ID NO: 28 - protein, 32 aa, herpes simplex virus 7
NKSLGVCPIRTQPRWSYYDSFSAVSEDNLGFL

SEQ ID NO: 29 - protein, 34 aa, herpes simplex virus 7
QPPSIPITVYYAVLERACRSVLLHAPSEAPQIVR

SEQ ID NO: 30 - protein, 31 aa, herpes simplex virus 7
ITQFILEHRARASCKYALPLRIPPAACLTSK

SEQ ID NO: 31 - protein, 35 aa, herpes simplex virus 7
AACLTSKAYQQGVTVDSIGMLPRFTPENQRTVALY

SEQ ID NO: 32 - protein, 28 aa, herpes simplex virus 7
TIAWYRMGDNCAIPITVMEYTECPYNKS

SEQ ID NO: 33 - protein, 28 aa, herpes simplex virus 7
APQIVRGASDEARKHTYNLTIAWYRMGD

SEQ ID NO: 34 - protein, 34 aa, herpes simplex virus 7; identical to SEQ ID NO: 7

SEQ ID NO: 35 - protein, 30 aa, herpes simplex virus 7
QRTVALYSLKIAGWHGPKPPYTSTLLPPEL

SEQ ID NO: 36 - protein, 32 aa, herpes simplex virus 7
NLPVLDQLTDPPGVKRVYHIQPSLEDPFQPPS

SEQ ID NO: 37 - protein, 21 aa, herpes simplex virus 7
IGGIAFWVRRRRSVAPKRLRL

SEQ ID NO: 38 - protein, 29 aa, herpes simplex virus 7
SKYALADPSLKMADPNRFRGKNLPVLDQL

SEQ ID NO: 39 - protein, 23 aa, herpes simplex virus 7
KYALADPSLKMADPNRFRGKNLP

SEQ ID NO: 40 - protein, 31 aa, herpes simplex virus 7
APQIPPNWHIPSIQDVATPHHAPAAPANPGL

SEQ ID NO: 41 - protein, 35 aa, herpes simplex virus 7
FRYVLQLQRNPMKALYPLTTKELKTSDPGGVGGEG

SEQ ID NO: 42 - protein, 40 aa, herpes simplex virus 7; identical to SEQ ID NO: 15

SEQ ID NO: 43 - protein, 30 aa, herpes simplex virus 7; identical to SEQ ID NO: 16

SEQ ID NO: 44 - protein, 32 aa, herpes simplex virus 7
SLAEAREMIRYMALVSAMERTEHKARKKGTSA

SEQ ID NO: 45 - protein, 33 aa, herpes simplex virus 7
ATHIKVGQPQYYQATGGFLIAYQPLLSNTLAEL

SEQ ID NO: 46 - protein, 28 aa, herpes simplex virus 7
HRGYFIFGGGYVYFEEYAYSHQLSRADV

SEQ ID NO: 47 - protein, 31 aa, herpes simplex virus 7
RADVTTVSTFIDLNITMLEDHEFVPLEVYTR

SEQ ID NO: 48 - protein, 23 aa, herpes simplex virus 7
NNDVRLTRDALEPCTVGHRGYFI

SEQ ID NO: 49 - protein, 22 aa, herpes simplex virus 7
PLLSNTLAELYVREYMREQDRK

SEQ ID NO: 50 - protein, 32 aa, herpes simplex virus 7
TKGVCRSTAKYVRNNLMTTAFHRDDHETDMEL

SEQ ID NO: 51 - protein, 38 aa, herpes simplex virus 7
PLREAPSANASVERIKTTSSIEFARLQFARLQFTYNHI

SEQ ID NO: 52 - protein, 27 aa, herpes simplex virus 7
DAQFYVCPPPTGATVVQFEQPRRCPTR

SEQ ID NO: 53 - protein, 34 aa, herpes simplex virus 7
GTSALLSSKVTNMVLRKRNKARYSPLHNEDEAGD

SEQ ID NO: 54 - protein, 27 aa, herpes simplex virus 7
SRPLVSFRYEDQGPLIEGQLGENNDVR

(19) United States
(12) Patent Application Publication
(10) Pub. No.: US 2008/0299140 A1
(43) Pub. Date: Dec. 4, 2008

(54) Immunogenic Composition and Peptide Sequences for Prevention and Treatment of an Hsv Condition

(75) Inventors: Bertrand Georges, Bauvin (FR); Helene Gras-Masse, Merignies (FR); Lbachir BenMohamed, Mission Viejo, CA (US); Anthony B. Nesburn, Mailbu, CA (US)

Correspondence Address: STOUT, UXA, BUYAN & MULLINS LLP, 4 VENTURE, SUITE 300, IRVINE, CA, 92618, US

(73) Assignee: The Regents of the University of California,, Oakland, CA (US)

(21) Appl. No.: 10/516,035

(22) PCT Filed: May 23, 2003
(86) PCT No.: PCT/IB03/03073

§ 371 Date: Oct. 28, 2005

Related U.S. Application Data
(63) Continuation-in-part of application No. 10/213,053, filed on Aug. 6, 2002, now abandoned.
(60) Provisional application No. 60/383,170, filed on May 24, 2002.

(51) Int. Cl. A61K 39/245; A61P 31/12

(52) U.S. Cl. 424/186.1; 424/231.1

(57) ABSTRACT

Immunogenic composition comprising at least one Herpes Simplex Virus type 1 (HSV-1) and/or type 2 (HSV-2) peptide sequence hearing at least one epitope from glycoprotein D (gD) and/or glycoprotein B (gB), a pharmaceutical carrier and/or a human compatible adjuvant, peptide sequences and uses thereof for prevention or treatment of an HSV condition.

The invention relates to immunogenic composition comprising at least one Herpes Simplex Virus type 1 (HSV-1) and/or type 2 (HSV-2) peptide sequence from glycoprotein D (gD) and/or glycoprotein B (gB), to said immunogenic composition for use as a medicament for prevention or treatment of an HSV condition, for diagnosis, and to peptide sequences and uses thereof.

The incidence of HSV has risen 30 percent since the 1970's. One in four adults has HSV, and there are an estimated one million new cases of this disease every year. HSV infections have been associated with a spectrum of clinical syndromes including cold sores, genital lesions, corneal blindness and encephalitis. The percentage of infected persons who are not cognizant of their own infection with HSV is over 50% largely because these individuals either do not express the classic symptoms (e.g., they remain asymptomatic) or because they dismiss HSV as merely an annoying itch or rash in those cases in which the disease has external manifestations. Additionally, HSV may be treated, but clinical research has yet to identify a cure. Therefore, one cannot rid himself of HSV once infected; one can merely attempt to control infection when it reactivates. However, despite the increase of HSV prevalence during the last three decades, an effective preventive or therapeutic vaccine that could help to control this epidemic is still not available.

There are two forms of herpes, commonly known as HSV-1 and HSV-2. Although HSV-1 is frequently associated with cold sores and HSV-2 with genital herpes, the viruses have many similarities and can infect either area of the body. HSV-specific B-cell and T-cell responses have been detected in humans during natural infection, yet latent infection and reactivation of HSV from peripheral ganglia and re-infection of the mucocutaneous tissues occurs frequently, causing recurrent ocular, labial or genital lesions. Other symptoms may include herpes keratitis, fever blisters, eczema herpeticum, cervical cancer, throat infections, rash, meningitis, nerve damage, and widespread infection in debilitated patients.

It is known that there is a high degree of homology between the sequence of HSV-1 and HSV-2. HSV-1 and HSV-2 comprise the most closely related pair of herpes-viruses for which complete genome sequences are presently known. The overall incidence of identical aligned nucleotides was superior to 80% in the protein-coding regions (Dolan A. et al., J. Virol., 1998, March; 72(3):2010-21; Bzik D J et al., Virology, 1986, December, 155(2):322-33). The homology is further confirmed on the basis of the observation of a lower attack rate of genital HSV-2 disease in subjects seropositive for HSV-1, suggesting that previous infection with HSV-1 confers protection against HSV-2 disease (Stanberry, New England J. Of Medicine, 2002, 347, p. 1652-61). The high homology in primary and secondary structure suggests a conserved, essential function for the gD and gB genes. In Long D. et al., Infect. Immun., 1984, February, 43(2):761-4, it appears that either gD-1 or gD-2 is a potential candidate for a subunit vaccine against herpetic infections.

A variety of traditional vaccine strategies have been explored to induce protective immunity against HSV and recurrences. Live, attenuated, and killed viruses have been shown to provide protective immunity in murine HSV model systems (H. E. Farrell et al., Journal of Virology, 1994, vol. 68, 927-932; K. Samoto et al., Cancer Gene Therapy, 2001, vol. 8, 269-277), and recent HSV vaccine development has focused on various forms of recombinant expressed virus coat glycoprotein. Immunization with Freund's adjuvant-emulsified viral coat glycoproteins of either HSV-1 or HSV-2 provides complete or partial protective immunity against infection with both types of HSV in murine models (J. E. Blaney et al., Journal of Virology, 1998, vol. 72, 9567-9574; H. Ghinsi et al., Journal of Virology, 1994, vol. 68, 2118-2126; E. Manikan et al., Journal of Virology, 1995, vol.69, 4711-4716; L. A. Morrison et al., Journal of Virology, 2001, vol. 75, 1195-1204; J. L. Sin et al., International Immunology, 1999, vol. 11, 1763-1773).

However, vaccine trials in human subjects with alum-absorbed gD protein (S. E. Straus et al., Lancet, 1994, vol. 343, 1460-1463) or with both gB and gD proteins emulsified with MF59 adjuvant have had only marginal success in reducing recurrent genital shedding and disease (P. R. Krause et al., Infectious Disease Clinics of North America, 1999, vol. 13, 61-81; S. E. Straus et al., Lancet, 1994, vol. 343, 1460-1463; S. E. Straus et al., Journal of Infectious Diseases, 1997, vol. 176, 1129-1134). The antibody response to these vaccines has been shown as similar to natural HSV infections, yet these vaccines have been thus far unable to induce a T helper type-1 (Th1)-like CD4⁺ T-cell response; this response is believed to be responsible for protection against HSV, at least in animal and human models (R. Stanberry et al., The New England Journal of Medicine, vol. 347, N^{o} 21, and Jeong-Im Sin et al., International Immunology, 1999, vol. 11, 1763-1773).

Among other challenges that have prevented the development of an effective HSV vaccine are heretofore unidentified immunogenic epitopes (i.e., the portion of an antigen (Ag) that binds to an antibody (Ab) paratope, or that is presented on the surface of Ag presenting cells to T-cells, thereby triggering an immune response), the uncertainty about the exact immune correlates of protection (L. Corey et al., New England Journal of Medicine, 1999, vol. 341, 1432-1438), and the development of an efficient and safe immunization strategy. Despite the emphasis on the Ab and CD8⁺ T cell responses (K. Goldsmith et al., Cornea, 1997, vol.16, 503-506; D. M. Koelle et al., Journal of Immunology, 2001, vol. 166, 4049-4058; R. Rouse et al., Journal of Virology, 1994, vol. 68, 5685-5689), there are growing evidences to support a pivotal role for the Th-1 subset of CD4⁺ T-cells in anti-herpes immunity (D. M. Koelle et al., Journal of Infectious Disease, 2000, vol. 182, 662-670; W. Kwok et al., Trends in Immunology, 2001, vol. 22, 583-588; Z. Mikloska et al., Journal of General Virology, 1998, vol. 79, 353-361; E. J. Novak et al., International Immunology, 2001, vol. 13, 799-806). Furthermore, induction, modulation and maintenance of a memory immune response to HSV, mediated by any kind of effector mechanism, require the activation of CD4⁺ T-cell help (S. Gangappa et al., European Journal of Immunology, 1999, vol. 29, 3674-3682; J. L. Sin et al., International Immunology, 1999, vol. 11, 1763-1773). Optimal activation of HSV-specific CD4⁺ Th-cells is therefore one rational for an effective vaccination protocol. Focusing T cell responses toward selected HSV-1 epitopes could be of value in the case of HSV, where CD4⁺ T cells directed to the immunodominant epitopes might have been inactivated and T-cells specific for subdominant epitopes might have escaped T cell tolerance (Y. Gao et al., Journal of General Virology, 1999, vol. 80, 2699-2704; E. J. Novak et al., International Immunology, 2001, vol. 13, 799-806).

Epitope based vaccine have received considerable attention for the development of prophylactic vaccines and immunotherapeutic strategies. The selection of appropriate epitopes should allow the immune system to be focused on immunodominant or subdominant epitopes of pathogens. Once the appropriate epitope have been defined, they can be delivered by various strategies including lipopeptides, viral vectors, synthetic particules, adjuvants, liposomes and naked oligonucleotides.

T-cells tend to recognize only a limited number of discrete epitopes on a protein Ag. In theory, numerous potential T-cell epitopes could be generated from a protein Ag. However, traditional approaches for identifying such epitopes from among the often hundreds or thousands of amino acids that cover the entire sequence of a protein Ag have used overlapping synthetic peptides (overlapping peptide method), which is inconvenient at best. In addition, progress on the mapping of T-cell epitopes has been slow due to reliance on studies of clones, an approach that generally involves extensive screening of T-cell precursors isolated from whole Ag-stimulated cells.

T helper epitopes are carried by peptides that are derived from proteins. T helper epitopes must bind to MHC class II at the surface of antigen presenting cells before being presented to CD4⁺ T lymphocytes.

In human populations, Major Histocompatibility Complex (MHC) class II molecules present a high degree of polymorphism. As an example, more than 200 different alleles have been described for the HLA-DRB1 locus. The polymorphism of Human Leucocyte Antigen (HLA) class II molecules represent a major limit in the identification of epitope with large population coverage. Interestingly, alleles are not equally distributed in defined populations where a limited number of alleles are preponderant and are present in the majority of individuals. As an example, in Caucasian populations, seven alleles (DRB1*0101, DRB1*0301, DRB1*0401, DRB1*0701, DRB1*1101, DRB1*1301, DRB1*1501) cover approximatively 60% of the HLA-DR phenotypic frequency. Moreover, HLA-DR53 (DRB4*0101) or HLA-DP4 (DPB1*0401) are over-represented alleles covering respectively 49 and 64% of the Caucasian population.

Most of the polymorphic residues reside in the peptide binding groove and evidently are responsible for MHC class II binding specificity. Mammalian Class II MHC proteins generally recognize amino-acid side chains embedded within a 9 residue stretch of a bound peptide (Brown, J. H., Nature. Jul. 1, 1993; 364(6432):33-9, Elferink, B. G., Hum Immunol. 1993 November; 38(3):201-5 Fremont, D. H., Science. May 17, 1996; 272(5264):1001-4).

The molecular basis of peptide/MHC class II interaction has been extensively studied. Five pockets called P1, P4, P6, P7 and P9 located in the binding groove of MHC class II molecules have been described and represent a common feature of all MHC class II molecules (Brown J H et al, Nature, 1993). Most pockets in the MHC class II binding groove are shaped by clusters of polymorphic residues and, thus, have distinct chemical and size characteristics in different HLA-DR alleles. Each MHC class II pocket can be characterized by their pocket profiles, a representation of the interaction of all natural amino acid residues with a given pocket. The capacity of a given peptide to bind a certain MHC class II molecules is the result of attracting and repelling forces between peptide side chains and residues lining the MHC binding site.

MHC class II molecule bind a large number of peptide ligand by using few peptide residues as anchor and considering that most of the binding energy implicated hydrogen bond between conserved residues of the MHC molecules and the peptide backbone. As a reciprocal consequence, it is well established that the binding of peptides to class II molecules may be promiscuous, that is a given peptide may bind several molecules and may even be recognized by the same T cell on differents class II molecules (Panina Bordignon, P., Eur J Immunol. 1989 December; 19(12):2237-42, Sinigaglia, F., Nature. Dec. 22-29, 1988; 336(6201):778-80). Promiscuous peptide binding to multiple MHC class II alleles were previously described and revealed two different mechanisms (i) peptides containing a unique and degenerate MHC class II binding register (ii) peptides containing several distinct but complementary MHC class II binding register (Hammer J, Cell. Jul. 16, 1993; 74(l):197-203., Sinigaglia Nature. Dec. 22-29, 1988; 336(6201):778-80., Hill C M, J Immunol. Mar. 15, 1994; 152(6):2890-8, Southwood S, J Immunol. Apr. 1, 1998; 160(7):3363-73). For all HLA-DR alleles, a large number of HLA-DP,-DQ and murine I-E alleles (Brown, J. H., Nature. Jul. 1, 1993; 364(6432):33-9 , Falk, 1994, Castelli, F. Journal of Immunology, Dec. 15, 2002, 169 (12); 6928-6934; Gosh P, nature, Nov. 30, 1995; 378 (6556), 457-462), a deep and hydrophobic anchor pocket play a dominant role at P1 position. Moreover, charged residues or bulky residue pointing to smaller binding pockets may also contribute in part to common criteria appear to be shared by mammals. As an example of the interspecies MHC class II peptide binding, mouse alleles and human alleles are all able to bind the class II-associated invariant chain peptide, which is basically identical in human and mouse. Indeed, the invariant chain peptide is characterized by having a methionine present at P1 position and at P4, P6 and P9 no strong anchors, but by the absence of inhibiting residues. As an example of the universality of CD4⁺ T cell epitopes, some malaria T-cell epitope were previously known to be recognized in association with most mouse and human MHC class II molecules (Sinigaglia F., Nature. Dec. 22-29, 1988; 336(6201):778-80).

Even if limited number of promiscuous CD4⁺ T cell epitopes have been previously described, their identification remains uncommon and difficult (Wilson, C. C., J. Virol. 2001. May, 75(9):4195-4207).

Several algorithms and database for MHC ligands were used to predict MHC binding peptides including motif based (SYFPEITHY) and matrix based (TEPITOPE=www.vaccinome.com, EPIPREDICT=www.epipredict.de, Propred=www.imtech.res.in/raghava/propred.), as described in Bian H. et al., Methods, 2003 Mar, 29(3):299-309; Raddrizzani L. et al., Brief Bioinform., 2000 May, 1, 2000(2):179-89; Sturniolo T. et al., Nat. Biotechnol., 1999 June, 17(6):555-61; de Lalla C. et al., J. Immunol., Aug. 15, 1999, 163(4):1725-9; Brusic V. et al., Bioinformatics, 1998, 14(2):121-30 ; Jung G. et al., Biologicals, 2001, September-December, 29(3-4):179-81; Singh H. et al., Bioinformatics, 2001 December, 17(12):1236-7; and Vordermeier M. et al., Infect. Immun., 2003 April, 71(4):1980-7.

Other, relatively laborious strategies have been used to identify small subsets of candidate epitopes by sequencing peptides eluted from purified MHC molecules from pathogen infected cells and then testing their MHC binding affinity. High affinity peptides are then tested for their ability to induce pathogen-specific T-cells. The major drawback of these approaches is the number of peptide sequences that need to be synthesized and tested, thus rendering them expensive, labor-intensive and time-consuming.

Yet even if T-cell epitopes could be accurately predicted and synthesized, peptide-based vaccines still- face limitations of weak immunogenicity, coupled with a paucity of sufficiently potent adjuvants that can be tolerated by humans. Large numbers of adjuvants are known to enhance both B-cell and T-cell responses in laboratory animals, but adjuvants compatible to humans are limited due to their toxic effects. The aluminum hydroxide salts (ALUM) are the only adjuvants widely used in human vaccines, but ALUM-adsorbed antigens preferentially induce Th2 responses as opposed to Th1 responses believed to be needed to increase the efficiency of a CD4⁺ T-cell immune response; especially advantageous in an HSV treatment.

In view of the drawbacks of the state of the art mentioned above, the Inventors set themselves the task of providing immunogenic compositions that induce a Th1 subset of a CD4⁺ T-cell immune response and that are safe and effective in humans and other mammals in treating and/or providing protective immunity against HSV infection, that is to say HSV-1 and HSV-2 infections.

These objectives are achieved through the creation of a new immunogenic composition comprising at least one HSV-1 and/or HSV-2 epitope containing peptide from gD and/or gB, a pharmaceutical carrier and/or a human compatible adjuvant, said epitope containing peptide having the capacity to bind on at least three alleles of humans HLA class II molecules having a frequency superior to 5% in a Caucasian population, with a binding activity less or equal to 1000 nanomolar.

Within the meaning of the present invention, “immunogenic composition” is to be taken as meaning that the composition is able to induce an immunity in animal and human models, that is to say the composition is able to prevent or treat a condition related to HSV.

These new immunogenic compositions allowing to obtain good results with MHC class II binding assay in human models must, in particular, meet the following criteria:
i) to induce a protective efficacy in the well established murine herpes model (Jeong-Im Sin, Int. Immnol. 1999, 11, 1763-1773), the guinea pig or the rabbit (Kern E R., DeClerque E and Walker R T edition, New York: plenum Press, 1987: 149-172),
ii) to generate potent Th1 subset CD4+ T-cell responses in mammals,
iii) to induce T-cell responses that are relevant to the native proteins.

The immunogenic composition according to the present invention can elicit potent CD4⁺ T-cell responses in animal and human models. While not wishing to be bound by any theory, it is believed that the immunogenic composition comprising epitope containing peptide induce the Th1 subset of T-cells by the selective expansion of CD4⁺ T-cells and stimulation of IL-2 and IFN-y; important cytokines in the elimination of HSV and the treatment of various other conditions. It is further believed that inducing the Th1 subset of T-cells may substantially increase the modulation and maintenance of a memory immune response to HSV. Therefore, a therapeutic basis for an effective treatment and vaccination against HSV may be the activation of HSV-specific CD4⁺ Th-cells with the immunogenic composition comprising epitope containing peptide of the present invention.

Within the meaning of the present invention, “epitope containing peptide” is to be taken as meaning that the peptide contains at least one epitope.

Within the meaning of the present invention, “prevent or treat” is to be taken as meaning, but is not limited to, ameliorating a disease, lessening the severity of its complications, preventing it from manifesting, preventing it from recurring, merely preventing it from worsening, mitigating an inflammatory response included therein, or a therapeutic effort to affect any of the aforementioned, even if such therapeutic effort is ultimately unsuccessful.

Within the meaning of the present invention, “human compatible adjuvant” is to be taken as meaning an adjuvant that is well-tolerated by the human recipients, and that can enhance a significant HSV-specific Th1 CD4⁺ T cell response.

Within the meaning of the present invention, “pharmaceutical carrier” is to be taken as meaning a pharmaceutically acceptable carrier that is compatible with the other ingredients of the formulation or composition and that is not toxic to the subjects to whom it is administered. One of such pharmaceutical carrier could be represented by lipidic tails such as those disclosed in the patent application published under number WO 02/20558.

The lipidic tail can be bound to the peptide of interest by acylation or chemoselective ligation, such as disclosed in D. Bonnet et al., J. Org. Chem., 2001, 66, 443-449; D. Bonnet et al., Tetrahedron Letters, 2000, 41, 10003-10007; Bourel-Bonnet L. et al., Bioconjug. Chem., 2003, March-April; 14(2):494-9; and D. Bonnet et al., J. Med Chem, 2001, 44, 468-471.

The lipidic tail can be bound to the peptide of interest by solid-phase synthesis, such as disclosed in the two following publications.

Brynestad K et al., J Virol. 1990 February, 64(2):680-5 discloses the influence of peptide acylation, liposome incorporation, and synthetic immunomodulators on the immunogenicity of a 1-23 peptide of gD of HSV-1. A peptide corresponding to residues 1 to 23 of gD of HSV-1 was chemically synthesized and coupled to a fatty acid carrier by standard Merrifield synthesis procedures. The resulting peptide-palmitic acid conjugate (acylpeptide) exhibited enhanced immunogenicity in mice as compared with that exhibited by the free form of the peptide.

As well, Watari E. et al., J Exp Med Feb. 1, 1987; 165(2):459-70, discloses the ability of peptides such as peptide corresponding to residues 1 to 23 of gD of HSV-1, covalently coupled to palmitic acid and incorporated into liposomes, to induce virus-specific T cell responses that confer protection against a lethal challenge of HSV-2. Thus, long-term protective immunity is achieved with a single immunization in the absence of neutralizing antibody when antigen is presented in this form. Furthermore, T cells but not serum from such immune mice can adoptively transfer this protection.

Within the meaning of the present invention, “the epitope having the capacity to bind on at least three alleles of humans HLA class II molecules having a frequency superior to 5% in a Caucasian population, with a binding activity less or equal to 1000 nanomolar” is to be taken as meaning peptide concentration allowing 50% inhibition of the binding of a reference tracer peptide.

For the selection of highly cross-reactive HLA-DR/HLA-DP binding peptides, the amino-acid sequences of gD and gB from HSV were scanned for the presence of HLA-DR motifs (TEPITOPE: www.vaccinome.com) and HLA-DP motifs (Castelli, F., J. Immunol., Dec. 15, 2002; 169(12):6928-34).

Specifically, 27 sequences between 15 to 40 amino-acids containing 9-residue core region comprised of a cluster of DR or DP motifs and several N- and C-terminal flanking amino-acids (between 3 to 6 amino-acids) were selected excluding signal peptide and highly hydrophobic transmembrane domain (THMMN=www.expasy.ch).

Twelve human and one murine MHC class II molecules have been selected to perform the MHC class II binding assays screening process with the HSV-derived peptides:(DR1=HLA-DR(α1*0101,α1*0101); DR15=HLA-DR(α1*0101,α1*1501); DR3=HLA-DR(α1*0101,α1*0301); DR4=HLA-DR(α1*0101,α1*0401), DR7=HLA-DR(α1*0101,α1*0701); DR11=HLA-DR(α1*0101,α1*1101); DR13=HLA-DR(α1*0101,α1*1301); DRB3=HLA-DR(α1*0101,α3*0101); DRB4=HLA-DR(α1*0101,α4*0101); DRB5=HLA-DR(α1*0101,α5*0101); DP401=HLA-DP(α1*0101,α1*0401); DP402=HLA-DR(α1*0101,α1*0402) and I-Ek). HLA class II molecules have been selected according to their very high phenotypic frequency in Caucasian population (see table in example 18 hereinafter). MHC class II binding assays have been largely used to identify potential promiscuous T cell epitopes within many proteins from different pathogens including virus, bacterial, parasites and from some tumor-specific antigens (Calvo-Calle, J. M., J Immunol. Aug. 1, 1997; 159(3):1362-73., Wilson, C. C., J Virol. 2001 May; 75(9):4195-207,Hammer, J., Adv Immunol. 1997; 66:67-100, Geluk, A., Eur J Immunol. 1992 January; 22(1):107-13, Zarzour, H. M., Cancer Res. Jan. 1, 2002; 62(1):213-8, Celis, E., Mol Immunol. 1994 December; 31(18):1423-30).

The strategy for resolving the problem of the present invention was thus to combine algorithms for MHC binding based on HLA-DR matrices, and binding assays for the experimental selection of epitope containing peptides able to bind with several HLA molecules and with mouse alleles.

Different studies suggest an IC50 of 1000 nM represents an affinity threshold associated with immunogenicity in the context of MHC class II molecules (Southwood S, J Immunol. Apr. 1, 1998; 160(7):3363-73, Wilson, C. C., J Virol. 2001 May; 75(9):4195-207). As a result of the 1000 nanomolar analysis, 25 highly cross-reactive HLA-DR/HLA-DP binding peptide to at least 5 different HLA class II molecules were identified Accordingly, a threshold of 800 nanomolar was used as a cut-off value for the epitope selection. As a result of this analysis, 23 highly cross-reactive HLA-DR/HLA-DP binding peptide to at least 5 different HLA class II molecules were identified.

According to one advantageous form of embodiment of the immunogenic composition according to the invention, the epitope containing peptide has the capacity to bind on at least five alleles of humans HLA class II molecules having a frequency superior to 5% in a Caucasian population, with a binding activity less or equal to 800 nanomolar.

According to another advantageous form of embodiment of the immunogenic composition according to the invention, the epitope containing peptide is selected from the group of peptide sequences consisting of SEQ ID N^{o}1 to SEQ ID N^{o}12, SEQ ID N^{o}14 to SEQ ID N^{o}25, SEQ ID N^{o}28 to SEQ ID N^{o}39, and SEQ ID N^{o}41 to SEQ ID N^{o}52, or fragments thereof.

Said peptide sequences are presented in Table Ic hereinafter. They include peptide sequences from HSV-1 and the corresponding peptide sequences from HSV-2, either from gD part, or from gB part. These peptide sequences, either alone or in combination with one another, may be useful in the treatment of HSV-1 and/or HSV-2 primary infections and recurrences and related disease conditions including, but in no way limited to, cold sores, genital lesions, corneal blindness, and encephalitis, and any other disease or pathological condition in which expansion of CD4⁺ T-cells, stimulation of IL-2 or IFN-y, and/or the induction of the Th-1 subset of T-cells may be desirable.

Within the meaning of the present invention, “fragments thereof” is to be taken as meaning that based on the peptide sequences SEQ ID N^{o}1 to SEQ ID N^{o}12, SEQ ID N^{o}14 to SEQ ID N^{o}25, SEQ ID N^{o}28 to SEQ ID N^{o}39, and SEQ ID N^{o}41 to SEQ ID N^{o}52, it is possible to add or delete a number of amino acids of said peptide sequences to get other peptide sequences that would have in the immunogenic composition the same activity defined in the present invention for said immunogenic composition. Said modified peptide sequences should preferably range from 9 amino-acids and 40 amino-acids.

As illustration, peptide sequence SEQ ID N^{o}11 has 29 amino-acids, and peptide sequence SEQ ID N^{o}12 has 23 amino-acids (deletion of 6 amino-acids). As represented hereinafter in Table VI of example 18, peptide sequence SEQ ID N^{o}11 having the capacity to bind on at least four (4) alleles of humans HLA class II molecules having a frequency superior to 5% in a Caucasian population, with a binding affinity less or equal to 1000 nanomolar. The fragment of peptide sequence SEQ ID N^{o}11, peptide sequence SEQ ID N^{o}12, having the capacity to bind on at least three (3) alleles of humans HLA class II molecules having a frequency superior to 5% in a Caucasian population, with a binding affinity less or equal to 1000 nanomolar.

It is possible to add as well amino-acids or other molecules which do not modify said activity of the based peptide sequences as defined in the present invention. As example, it is possible to add amino-acids such as arginine or lysine, for an improved solubility of the peptide, or to replace cysteine residues by modified amino-acid residues such as alanine, serine or leucine, provided no loss of binding activity of the based peptide sequences as defined in the present invention.

According to another advantageous form of embodiment of the immunogenic composition according to the invention, the immunogenic composition comprises a combination of 2 to 8 epitope containing peptides.

It is to be understood that the peptide sequences described herein, either alone or in any suitable combination, either with one another or with additional peptide sequences not specifically enumerated herein, would be readily recognized by one of skill in the art. gD and gB peptide sequences or proteins, or fragment thereof, from HSV-1 and HSV-2 according to the present invention, are conventionally administered in an immunogenic composition to ameliorate the symptoms of HSV, and to thereby slow or halt the spread of HSV disease; although the gD and gB peptide sequences of the present invention may additionally be used in the prevention of HSV infection (e.g., as a prophylactic vaccine). Thus, in embodiments of the present invention, the peptide sequences may be administered in a multi-component immuno-therapeutic (i.e., to treat the disease) and/or an immuno-prophylactic (i.e., to prevent the disease) composition as vaccine, effective against HSV. In particular, the gD and gB peptide sequences present in the immunogenic composition according to the present invention may provide at least partial, and in some cases full protective immunity to HSV, and may thereby function as a preventative vaccination.

In a particularly advantageous manner, the immunogenic composition according to the invention, comprises a combination of 3 to 7 epitope containing peptides from gD HSV-1 selected from the group of peptide sequences consisting of SEQ ID N^{o}2, SEQ ID N^{o}5, SEQ ID N^{o}7, SEQ ID N^{o}8, SEQ ID N^{o}10, SEQ ID N^{o}11 and SEQ ID N^{o}12, preferably a combination of 3 to 5 epitope containing peptides selected from the group of peptide sequences consisting of SEQ ID N^{o}2, SEQ ID N^{o}7, SEQ ID N^{o}8, SEQ ID N^{o}10, and SEQ ID N^{o}11, and more preferably a combination of 4 epitope containing peptides selected from the group of peptide sequences consisting of SEQ ID N^{o}2, SEQ ID N^{o}7, SEQ ID N^{o}8 and SEQ ID N^{o}10, and/or the corresponding gD HSV-2 epitope containing peptides, or combinations of said gD HSV-1 and gD HSV-2 epitope containing peptides.

Within the meaning of the present invention, “corresponding gD HSV-2 epitope containing peptides” is to be taken as meaning that the peptide sequence of HSV-1 present a high degree of homology with the peptide sequence of HSV-2.

In the immunogenic composition according to the present invention, any of the peptide sequences represented by SEQ ID N^{o}2, SEQ ID N^{o}5, SEQ ID N^{o}7, SEQ ID N^{o}8, SEQ ID N^{o}10, SEQ ID N^{o}11 and SEQ ID N^{o}12, any peptide sequences including one or more of the peptide sequences represented by SEQ ID N^{o}2, SEQ ID N^{o}5, SEQ ID N^{o}7, SEQ ID N^{o}8, SEQ ID N^{o}10, SEQ ID N^{o}11 and SEQ ID N^{o}12, any portion of the peptide sequences represented by SEQ ID N^{o}2, SEQ ID N^{o}5, SEQ ID N^{o}7, SEQ ID N^{o}8, SEQ ID N^{o}10, SEQ ID N^{o} 11 and SEQ ID N^{o}12 or combinations thereof may be incorporated into said immunogenic composition effective in the prevention and/or treatment of HSV.

It is to be understood that the immunogenic composition according to the present invention may comprise the precedent cited peptide sequences, as well as the peptide sequences from HSV-1 and/or HSV-2 gB, as indicated in table 1c. The man skilled in the art been able to choose those peptide sequences, knowing the result of the MHC binding and the homology percentage between the peptide sequences from HSV-1 and HSV-2.

In alternate embodiments of the present invention, one may implement one or more of the peptide sequences of the present invention, but, to obtain a desired clinical result, one may not need to utilize the entire sequence. In fact, a portion of one or more of the peptides represented by SEQ ID N^{o}2, SEQ ID N^{o}5, SEQ ID N^{o}7, SEQ ID N^{o}8, SEQ ID N^{o}10, SEQ ID N^{o}11 and SEQ ID N^{o}12 may be clinically effective. In still further embodiments of the present invention, one may include one or more of the peptide sequences of the present invention represented by SEQ ID N^{o}2, SEQ ID N^{o}5, SEQ ID N^{o}7, SEQ ID N^{o}8, SEQ ID N^{o}10, SEQ ID N^{o}11 and SEQ ID N^{o}12 in a larger protein molecule. Doing so may be advantageous for any number of reasons, as will be readily recognized by one of skill in the art. Including one of the peptide sequences in such a larger molecule is also contemplated as being within the scope of the present invention.

In a particularly advantageous manner, the corresponding HSV-2 epitope containing peptides present an homology of the peptide sequence with the HSV-1 epitope containing peptide of at least 70%, preferably at least 80%, more preferably at least 90%.

There are various reasons why one might wish to administer an immunogenic composition of the present invention comprising a combination of epitope containing peptides rather than a single epitope containing peptide. Depending on the particular peptide sequence that one uses, an immunogenic composition might have superior characteristics as far as clinical efficacy, solubility, absorption, stability, toxicity and patient acceptability are concerned. It should be readily apparent to one of ordinary skill in the art how one can formulate an immunogenic composition of any of a number of combinations of peptide sequences of the present invention. There are many strategies for doing so, any one of which may be implemented by routine experimentation. For example, one can survey specific patient MHC restriction or test different combinations, as illustrated in the ensuing example 13.

The immunogenic composition comprising at least one epitope containing peptide of the present invention may be administered as a single agent therapy or in addition to an established therapy, such as inoculation with live, attenuated, or killed virus, or any other therapy known in the art to treat HSV.

The appropriate dosage of the epitope containing peptide or peptide sequence of the immunogenic composition of the invention may depend on a variety of factors. Such factors may include, but are in no way limited to, a patient's physical characteristics (e.g., age, weight, sex), whether the composition is being used as single agent or adjuvant therapy, the type of MHC restriction of the patient, the progression (i.e., pathological state) of the HSV infection, and other factors that may be recognized by one skilled in the art. In general, a peptide sequence or combination of peptide sequence may be administered to a patient in an amount of from about 50 micrograms to about 5 mg; dosage in an amount of from about 50 micrograms to about 500 micrograms is especially preferred.

In a particularly advantageous manner, the immunogen composition includes an adjuvant; most preferably, Montanide ISA720 (M-ISA-720; available from Seppic, Fairfield, N.J.), an adjuvant based on a natural metabolizable oil. As further described in the ensuing examples, M-ISA-720 was found to enhance a significant HSV-specific Th1 CD4⁺ T-cell response, and the subcutaneous injection of vaccine formulated with the same was well-tolerated by recipients. Immunogenic composition of the present invention preferably include from about 15 μl to about 25 μL M-ISA-720.

Immunogenic composition of the invention may be prepared by combining at least one epitope containing peptide with a pharmaceutically acceptable liquid carrier, a finely divided solid carrier, or both.

Suitable such carriers may include, for example, water, alcohols, natural or hardened oils and waxes, calcium and sodium carbonates, calcium phosphate, kaolin, talc, lactose, combinations thereof and any other suitable carrier as will be recognized by one of skill in the art.

In a particularly advantageous manner, the carrier is present in an amount of from about 10 μl (micro-liter) to about 100 μl.

In various embodiments, immunogenic composition according to the invention may be combined with one or more additional components that are typical of pharmaceutical formulations such as vaccines, and can be identified and incorporated into the immunogenic composition of the present invention by routine experimentation. Such additional components may include, but are in no way limited to, excipients such as the following: preservatives, such as ethyl-p-hydroxybenzoate; suspending agents such as methyl cellulose, tragacanth, and sodium alginate; wetting agents such as lecithin, polyoxyethylene stearate, and polyoxyethylene sorbitan mono-oleate; granulating and disintegrating agents such as starch and alginic acid; binding agents such as starch, gelatin, and acacia; lubricating agents such as magnesium stearate, stearic acid, and talc; flavoring and coloring agents; and any other excipient conventionally added to pharmaceutical formulations.

In a particularly advantageous manner, the immunogenic composition according to the invention further comprises an additional component selected from the group consisting of a vehicle, an additive, an excipient, a pharmaceutical adjunct, a therapeutic compound or agent useful in the treatment of HSV and combinations thereof.

One may administer an immunogenic composition of the present invention by any suitable route, which may include, but is not limited to, systemic injections (e.g., subcutaneous injection, intradermal injection, intramuscular injection, intravenous infusion) mucosal administrations (e.g., nasal, ocular, oral, vaginal and anal formulations), topical administration (e.g., patch delivery), or by any other pharmacologically appropriate technique. Vaccination protocols using a spray, drop, aerosol, gel or sweet formulation are particularly attractive and may be also used. The immunogenic composition may be administered for delivery at a particular time interval, or may be suitable for a single administration. In those embodiments wherein the immunogenic composition of the present invention is formulated for administration at a delivery interval, it is preferably administered once every 4 to 6 weeks.

In a particularly advantageous manner, the immunogenic composition according to the invention is formulated to be administered by systemic injection, particularly by subcutaneous injection.

Another object of the invention is an immunogenic composition for use as a medicament. The different way of administration have been described previously.

Still another object of the invention is an immunogenic composition according to the present invention for the manufacture of a medicament for prevention or treatment of a condition selected from the group consisting of HSV-1 primary infections, HSV-1 recurrences, HSV-2 primary infection, HSV-2 recurrences, cold sores, genital lesions, corneal blindness, and encephalitis, a condition in which a stimulation of IL-2 and IFN-γ is desirable and in which the induction of the Th-1 subset of T-cells is desirable.

Still another object of the invention is an HSV-1 or HSV-2 peptide sequence bearing at least one epitope, or fragment thereof, wherein said peptide sequence is represented by one peptide sequence selected from the group consisting of SEQ ID N^{o}1 to SEQ ID N^{o}11, SEQ ID N^{o}14 to SEQ ID N^{o}52, and use of said peptide sequence(s) for the manufacture of a medicament according to the invention, for treating or preventing a condition related to HSV-1 and/or HSV-2, and for the manufacture of a diagnosis reagent.

The administration of said medicament has been described previously.

As diagnosis reagent, the peptide sequences according to the present invention could be under a multimeric complex form, and preferably under a tetramer complex form, as described in the patent application filed under FR 0209874.

In addition to the preceding provisions, the invention includes yet others which will emerge from the description that follows, which refers to examples of implementation of the immunogenic composition according to the present invention, as well as to the annexed drawings, wherein:

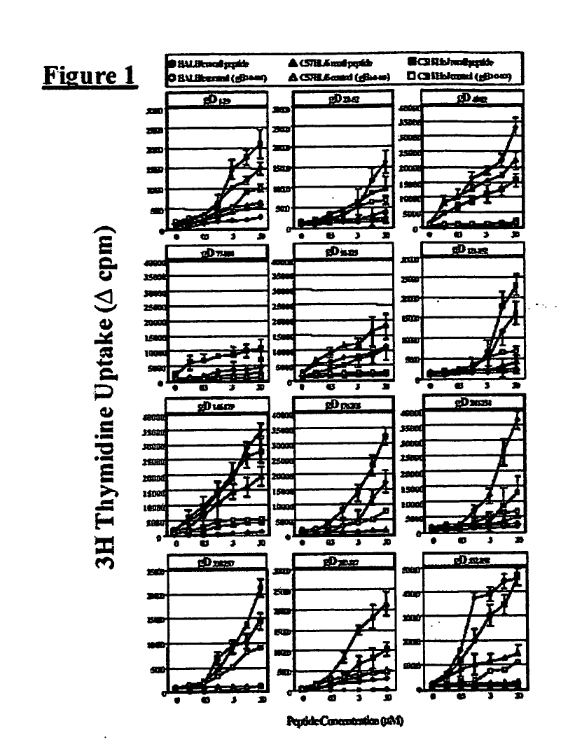

FIG. 1 is a graphical representation of the proliferative responses generated by HSV-1 gD peptide sequences, peptide sequence concentration was measured in μM.

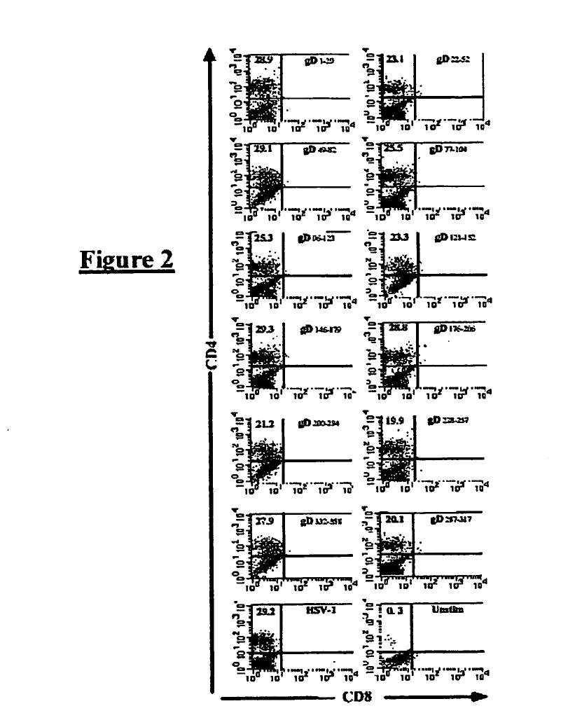

FIG. 2 depicts a fluorescent activated cell sorter (FACS) analysis of stimulated cells graphically depicted in FIG. 1 in accordance with an embodiment of the present invention. Most responding cells were of CD4⁺ phenotype.

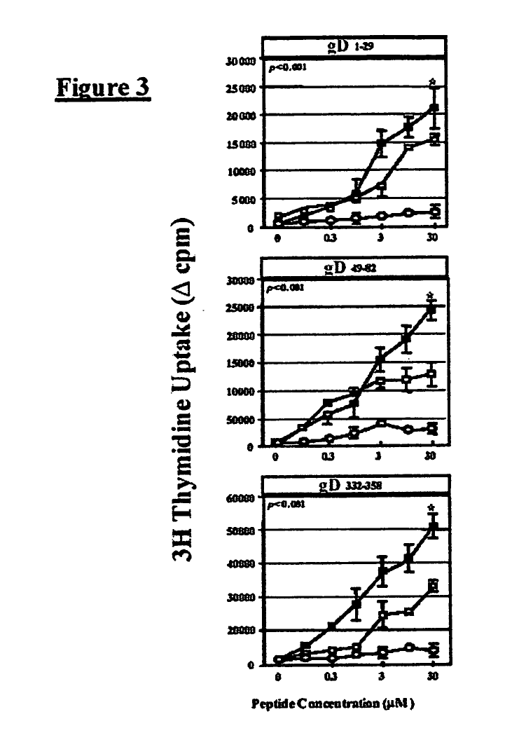

FIG. 3 is a graphical representation of the proliferative responses generated by each of the dominant HSV-1 gD peptide sequence predicted from the TEPITOPE algorithm in accordance with an embodiment of the present invention. Peptide sequence concentration was measured in μM.

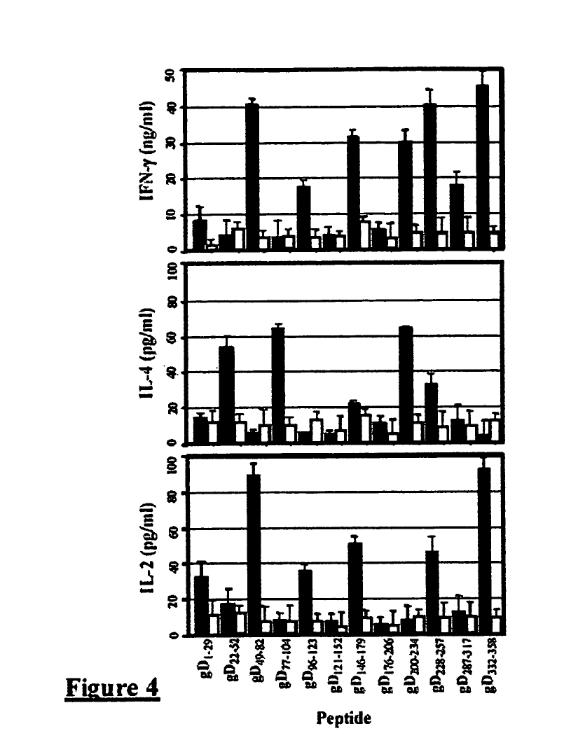

FIG. 4 is a graphical representation of cytokine secretion elicited by HSV-1 gD peptide.

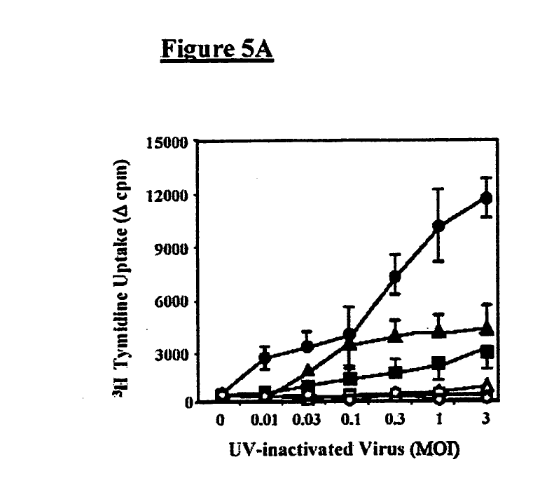

FIG. 5 is a graphical representation of ³H Thymidine uptake in accordance with an embodiment of the present invention. FIG. 5A depicts ³H Thymidine uptake by ultraviolet-inactivated HSV-1, and FIG. 5B depicts ³H Thymidine uptake by ultraviolet-inactivated HSV-1 comparing HSV infected dendritic cells and HSV mock infected dendritic cells.

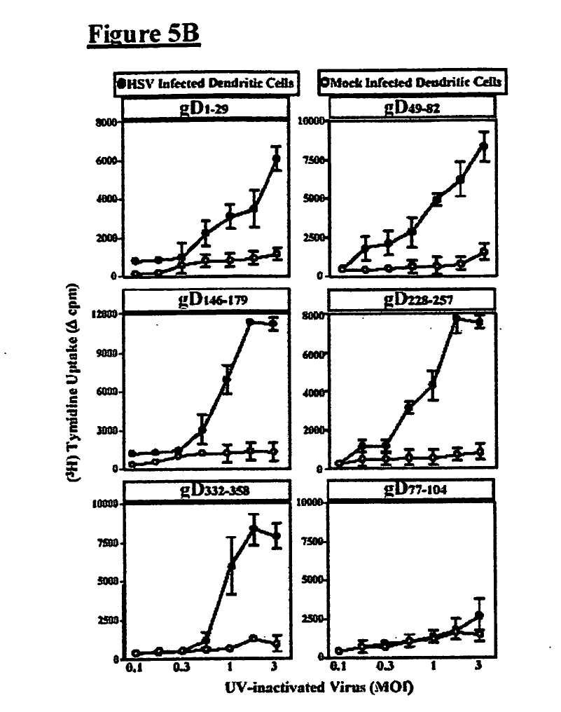

FIG. 6 is a graphical representation of ³H Thymidine uptake by HSV-1 gD peptides comparing HSV infected dendritic cells and HSV mock infected dendritic cells in accordance with an embodiment of the present invention.

It should be clearly understood, however, that these examples are given solely by way of illustration of the object of the invention, of which they are in no way limitative.

Even if the examples illustrate the activity of some immunogenic composition comprising HSV-1 peptide sequences from gD and gB, the present invention encompass immunogenic composition comprising the corresponding HSV-2 peptide sequences, based on the following homology in Table Ia and Ib.

TABLE Ia

| | % homology |
|---|---|
| | with |
| HSV-1 gD | corresponding |
| peptides | HSV-2 peptide |
| HSV1 33 | 95% |
| HSV1 36 | 94% |
| HSV1 38 | 81% |
| HSV1 37 | 83% |
| HSV1 41 | 89% |
| HSV1 32 | 75% |
| HSV1 34 | 100% |
| HSV1 40 | 93% |
| HSV1 31 | 84% |
| HSV1 39 | 62% |
| HSV1 30 | 90% |
| HSV1 29 | 87% |
| HSV1 35 | 81% |

TABLE Ib

| | % homology |
|---|---|
| | with |
| HSV-1 gB | corresponding |
| peptides | HSV-2 peptide |
| HSV1 8 | 69% |
| HSV1 6 | 100% |
| HSV1 3 | 100% |
| HSV1 1 | 94% |
| HSV1 2 | 94% |
| HSV1 14 | 89% |
| HSV1 7 | 97% |
| HSV1 13 | 78% |
| HSV1 4 | 86% |
| HSV1 5 | 94% |
| HSV1 11 | 79% |
| HSV1 10 | 96% |
| HSV1 9 | 57% |
| HSV 12 | 89% |

### EXAMPLE 1

### T-cell Epitope Prediction

The gD and gB protein sequences from HSV-1 and HSV-2 were loaded into prediction software (TEPITOPE) and scanned for the presence of HLA-DP motifs (Castelli, F., J. Immunol., Dec. 15, 2002; 169(12):6928-34) to predict promiscuous epitopes. The TEPITOPE algorithm is a WINDOWS (Microsoft Corporation, Redmond, Wash.) application that is based on 25 quantitative matrix-based motifs that cover a significant part of human, HLA class II peptide binding specificity. Starting from any protein sequence, the algorithm permits the prediction and parallel display of ligands for each of the 25 HLA-DR alleles. The TEPITOPE prediction threshold, which was set at 10%, predicted fifty four regions (SEQ ID NOS:1-54).

The results are given in the following Table Ic.

TABLE Ic

Peptide sequence bearing potential T-cell epitopes
identified within the HSV-1 and HSV-2 gD and gB using the
TEPITOP algorithm.

| | |
|---|---|
| SEQ | |
| ID | Peptides |

| | | |
|---|---|---|
| No | AA* | Sequences |

| | | | | |
|---|---|---|---|---|
| 1 | HSV1 33 | 32 | gD₁₂₁₋₁₅₂ | NKSLGACPIRTQPRWNYYDSFSAVSEDNLGFL |
| 2 | HSV1 36 | 34 | qD₄₉₋₈₂ | QPPSLPITVYYAVLERACRSVLLNAPSEAPQIVR |
| 3 | HSV1 38 | 31 | gD₁₇₆₋₂₀₆ | ITQFILEHRAKGSCKYALPLRIPPSACLSPQ |
| 4 | HSV1 37 | 35 | gD₂₀₀₋₂₃₄ | SACLSPQAYQQGVTVDSIGMLPRFIPENQRTVAVY |
| 5 | HSV1 41 | 28 | gD₉₆₋₁₂₃ | TIAWFRMGGNCAIPITVMEYTECSYNKS |
| 6 | HSV1 32 | 28 | gD₇₇₋₁₀₄ | APQIVRGASEDVRKQPYNLTIAWFRMGG |
| 7 | HSV1 34 | 34 | gD₁₄₆₋₁₇₉ | EDNLGFLMHAPAFETAGTYLRLVKINDWTEITQF |
| 8 | HSV1 40 | 30 | gD₂₂₈₋₂₅₇ | QRTVAVYSLKIAGWHGPKAPYTSTLLPPEL |
| 9 | HSV1 31 | 32 | gD₂₂₋₅₂ | DLPVLDQLTDPPGVRRVYHIQAGLPDPFQPPS |
| 10 | HSV1 39 | 27 | gD₃₃₂₋₃₅₈ | ICGVYWMRRHTQKAPKRIRLPHIRED |
| 11 | HSV1 30 | 29 | gD₀₋₂₈ | SKYALVDASLKMADPNRFRGKDLPVLDQL |
| 12 | HSV1 29 | 23 | gD₁₋₂₃ | KYALVDASLKMADPNRFRGKDLP |
| 13 | HSV1 35 | 31 | gD₂₈₇₋₃₁₇ | APQIPPNWHIPSIQDAATPYHPPATPNNMGL |
| 14 | HSV1 8 | 35 | gB₇₆₅₋₇₉₉ | FRYVMRLQSNPMKALYPLTTKELKNPTNPDASGEG |
| 15 | HSV1 6 | 40 | gB₂₄₃₋₂₈₂ | VEEVDARSVYPYDEFVLATGDFVYMSPFYGYREGSHTEHT |
| 16 | HSV1 3 | 30 | gB₁₁₁₋₁₄₀ | NYTEGIAVVFKENIAPYKFKATMYYKDVTV |
| 17 | HSV1 1 | 32 | gB₈₀₉₋₈₄₀ | KLAEAREMIRYMALVSAMERTEHKAKKKGTSA |
| 18 | HSV1 2 | 33 | gB₄₀₁₋₄₃₃ | ATHIKVGQPQYYLANGGFLIAYQPLLSNTLAEL |
| 19 | HSV1 14 | 28 | gB₆₀₇₋₆₃₄ | HRRYFTFGGGYVYFEEYAYSHQLSRADI |
| 20 | HSV1 7 | 31 | gB₆₃₁₋₆₆₁ | RADITTVSTFIDLNITMLEDHEFVPLEVYTR |
| 21 | HSV1 13 | 23 | gB₅₉₀₋₆₁₂ | NNELRLTRDAIEPCTVGHRRYFT |
| 22 | HSV1 4 | 22 | gB₄₂₄₋₄₄₅ | PLLSNTLAELYVREHLREQSRK |
| 23 | HSV1 5 | 32 | gB₁₇₃₋₂₀₄ | AKGVCRSTAKYVRNNLETTAFHRDDHETDMEL |
| 24 | HSV1 11 | 36 | gB₄₅₃₋₄₈₃ | PPGASANASVERIKTTSSIEFARLQFARLQFTYNHI |
| 25 | HSV1 10 | 27 | gB₈₀₋₁₀₆ | DANFYVCPPPTGATVVQFEQPRRCPTR |
| 26 | HSV1 9 | 34 | gB₈₃₇₋₈₇₀ | GTSALLSAKVTDMVMRKRRNTNYTQVPNKDGDAD |
| 27 | HSV1 12 | 27 | gB₅₆₈₋₅₉₄ | SRPLVSFRYEDQGPLVEGQLGENNELR |
| 28 | HSV2 33 | 32 | gD₁₂₁₋₁₅₂ | NKSLGVCPIRTQPRWSYYDSFSAVSEDNLGFL |
| 29 | HSV2 36 | 34 | gD₄₉₋₈₂ | QPPSIPITVYYAVLERACRSVLLHAPSEAPQIVR |
| 30 | HSV2 38 | 31 | gD₁₇₆₋₂₀₆ | ITQFILEHRARASCKYALPLRIPPAACLTSK |
| 31 | HSV2 37 | 35 | gD₂₀₀₋₂₃₄ | AACLTSKAYQQGVTVDSIGMLPRFTPENQRTVALY |
| 32 | HSV2 41 | 28 | gD₉₆₋₁₂₃ | TIAWYRMGDNCAIPITVMEYTECPYNKS |
| 33 | HSV2 32 | 28 | gD₇₇₋₁₀₄ | APQIVRGASDEARKHTYNLTIAWYRMGD |
| 34 | HSV2 34 | 34 | gD₁₄₆₋₁₇₉ | EDNLGFLMHAPAFETAGTYLRLVKINDWTEITQF |
| 35 | HSV2 40 | 30 | gD₂₂₈₋₂₅₇ | QRTVALYSLKIAGWHGPKPPYTSTLLPPEL |
| 36 | HSV2 31 | 32 | gD₂₂₋₅₂ | NLPVLDQLTDPPGVKRVYHIQPSLEDPFQPPS |
| 37 | HSV2 39 | 21 | gD₃₃₂₋₃₅₈ | IGGIAFWVRRRRSVAPKRLRL |
| 38 | HSV2 30 | 29 | gB₀₋₂₈ | SKYALADPSLKMADPNRFRGKNLPVLDQL |
| 39 | HSV2 29 | 23 | gB₁₋₂₃ | KYALADPSLKMADPNRFRGKNLP |
| 40 | HSV2 35 | 31 | gB₂₈₇₋₃₁₇ | APQIPPNWHIPSIQDVATPHHAPAAPANPGL |
| 41 | HSV2 8 | 35 | gB₇₇₀₋₈₀₄ | FRYVLQLQRNPMKALYPLTTKELKTSDPGGVGGEG |
| 42 | HSV2 6 | 40 | gB₂₄₆₋₂₈₅ | VEEVDARSVYPYDEFVLATGDFVYMSPFYGYREGSHTEHT |
| 43 | HSV2 3 | 30 | gB₁₁₄₋₁₄₃ | NYTEGIAVVFKENIAPYKFKATMYYKDVTV |
| 44 | HSV2 1 | 32 | gB₈₁₇₋₈₄₈ | SLAEAREMIRYMALVSAMERTEHKARKKGTSA |
| 45 | HSV2 2 | 33 | gB₄₀₄₋₄₃₆ | ATHIKVGQPQYYQATGGFLIAYQPLLSNTLAEL |
| 46 | HSV2 14 | 28 | gB₆₁₂₋₆₃₉ | HRGYFIFGGGYVYFEEYAYSHQLSRADV |
| 47 | HSV2 7 | 31 | gB₆₃₆₋₆₆₆ | RADVTTVSTFIDLNITMLEDHEFVPLEVYTR |
| 48 | HSV2 13 | 23 | gB₅₉₅₋₆₁₇ | NNDVRLTRDALEPCTVGHRGYFI |
| 49 | HSV2 4 | 22 | gB₄₂₇₋₄₄₈ | PLLSNTLAELYVREYMREQDRK |
| 50 | HSV2 5 | 32 | gB₁₇₆₋₂₀₇ | TKGVCRSTAKYVRNNLNTTAFHRDDHETDMEL |
| 51 | HSV2 11 | 38 | gB₄₅₆₋₄₈₈ | PLREAPSANASVERIKTTSSIEFARLQFARQFTYNHI |
| 52 | HSV2 10 | 27 | gB₈₃₋₁₁₉ | DAQFYVCPPPTGATVVQFEQPRRCPTR |
| 53 | HSV2 9 | 34 | gB₈₄₅₋₈₇₈ | GTSALLSSKVTNMVLRKRNKARYSPLHNEDEAGD |
| 54 | HSV2 12 | 27 | gB₅₅₆₋₅₉₉ | SRPLVSFRYEDQGPLIEGQLGENNDVR |

*amino-acids

### EXAMPLE 2

### Synthesis of Peptides

A total of 27 gD and gB peptides (SEQ ID N^{o}1-27), each consisting of 21 to 40 amino acids, were synthesized by BioSource International (Hopkinton, Mass.) on a 9050 Pep Synthesizer Instrument using solid phase peptide synthesis (SPPS) and standard F-moc technology (PE Applied Biosystems, Foster City, Calif.). Peptides were cleaved from the resin using Trifluoroacetic acid:Anisole:Thioanisole:Anisole:EOT:Water (87.5:2.5:2.5:2.5:5%) followed by ether extraction (methyl-f-butyl ether) and lyophilization. The purity of peptides was greater than 90%, as determined by reversed phase high performance liquid chromatography (RP-HPLC) (VYDAC C18) and mass spectrometry (VOYAGER MALDI-TOF System). Stock solutions were made at 1 mg/ml in water, except for peptide gD₁₄₆₋₁₇₉ (SEQ ID N^{o}7) that was solubilized in phosphate buffered saline (PBS). All peptides were aliquoted, and stored at −20° C. until assayed. Studies were conducted with the immunogen emulsified in M-ISA-720 adjuvant (Seppic, Fairfield, N.J.) at a 3:7 ratio and immediately injected into mice.

### EXAMPLE 3

### Preparation of Herpes Simplex Virus Type 1

The McKrae strain of HSV-1 was used in this study. The virus was triple plaque purified using classical virology techniques. UV-inactivated HSV-1 (UV-HSV-1) was made by exposing the live virus to a Phillips 30 W UV bulb for 10 min at a distance of 5 cm. HSV inactivation in this manner was ascertained by the inability of UV-HSV-1 to produce plaques when tested on vero cells.

### EXAMPLE 4

### Immunization in Animal Models

Six to eight week old C57BL/6 (H-2b), BALB/c (H-2^{d}), and C3H/HeJ (H-2^{k}) mice (The Jackson Laboratory, Bar Harbor, Me.) were used in all experiments. Groups of five mice per strain, were immunized subcutaneously with peptides in M-ISA 720 adjuvant on days 0 and 21. In an initial experiment the optimal dose response to peptide gD₀₋₂₈ was investigated and no significant differences were found among doses of 50, 100 and 200 μg. Subsequent experiments used 100 μg (at day 0) and 50 μg (at day 21) of each peptide in a total volume of 100 μl. Under identical conditions control mice received the adjuvant alone, for control purposes.

### EXAMPLE 5

### Peptide-specific T-cell Assay

Twelve days after the second immunization, spleen and inguinal lymph nodes (LN) were removed and placed into ice-cold serum free HL-1 medium supplemented with 15 mM HEPES, 5×10⁻⁵ M β-mercaptoethanol, 2 mM glutamine, 50 U of penicillin and 50 μg of streptomycin (GIBCO-BRL, Grand Island, N.Y.) (complete medium, CM). The cells were cultured in 96-well plates at 5×10⁵ cells/well in CM, with recall or control peptide at 30, 10, 3, 1, or 0.3 μg/ml concentration, as previously described in (BenMohamed et al., 2000 and 2002). The cell suspensions were incubated for 72 h at 37° C. in 5% CO₂. One μCi (micro-curie) of (³H)-thymidine (Dupont MEN, Boston, Mass.) was added to each well during the last 16 h of culture. The incorporated radioactivity was determined by harvesting cells onto glass fiber filters and counted on a Matrix 96 direct ionization-counter (Packard Instruments, Meriden, Conn.). Results were expressed as the mean cpm of cell-associated (³H)-thymidine recovered from wells containing Ag minus the mean cpm of cell-associated (³H)-thymidine recovered from wells without Ag (A cpm) (average of triplicate). The Stimulation Index (SI) was calculated as the mean cpm of cell-associated (³H)-thymidine recovered from wells containing Ag divided by the mean cpm of cell-associated (³H)-thymidine recovered from wells without Ag (average of triplicate). For all experiments the irrelevant control peptide gB₁₄₁₋₁₆₅ and the T-cell mitogen Concanavalin A (ConA) (Sigma, St. Louis, Mo.) were used as negative and positive controls, respectively. Proliferation results were confirmed by repeating each experiment twice. A T-cell proliferative response was considered positive when A cpm>1000 and SI>2.

### EXAMPLE 6

### Cytokine Analysis

T-cells were stimulated with either immunizing peptides (10 μg/ml), the irrelevant control peptide (10 μg/ml), UV-inactivated HSV-1 (MOI=3), or with ConA (0.5 μg/ml) as a positive control. Culture media were harvested 48 h (for IL-2) or 96 h (for IL-4 and IFN-γ) later and analyzed by specific sandwich ELISA following the manufacturer's instructions (PharMingen, San Diego, Calif.).

### EXAMPLE 7

### Flow Cytometric Analysis

The gD peptide stimulated T-cells were phenotyped by double staining with anti-CD4⁺ and anti-CD8⁺ monoclonal antibodies (mAbs) and analyzed by FACS. After 4 days stimulation with 10 μM of each peptide, one million cells were washed in cold PBS-5% buffer and incubated with phycoerythrin (PE) anti-CD4 (Pharmingen, San Diego, Calif.) or with FITC anti-CD8⁺ (Pharmingen, San Diego, Calif.) mAbs for 20-30 min on ice. Propidium iodide was used to exclude dead cells. For each sample, 20,000 events were acquired on a FACSCALIBUR and analyzed with CELLQUEST software (Becton Dickinson, San Jose, Calif.), on an integrated POWER MAC G4 (Apple Computer, Inc., Cupertino, Calif.).

### EXAMPLE 8

### Derivation of Bone Marrow Dendritic Cells

Murine bone marrow-derived dendritic cells (DC) were generated using a modified version of the protocol as described previously in (BenMohamed et al., 2002). Briefly, bone marrow cells were flushed out from tibias and femurs with RPMI-1640, and a single cell suspension was made. A total of 2×10⁶ cells cultured in 100-P tissue dishes containing 10 ml of RPMI-1640 supplemented with 2 mM glutamine, 1% non-essential amino acids (Gibco-BRL), 10% fetal calf serum, 50 ng/ml granulocyte macrophage colony stimulatory factor (GM-CSF) and 50 ng/ml IL-4 (PeproTech Inc, Rocky Hill, N.J.). Cells were fed with fresh media supplemented with 25 ng/ml GM-CSF and 25 ng/ml IL-4 every 72 hrs. After 7 days of incubation, this protocol yielded 50-60×10⁶ cells, with 70 to 90% of the non-adherent-cells acquiring the typical morphology of DC. This was routinely confirmed by FACS analysis of CD11c, class II and DEC-205 surface markers of DC.

### EXAMPLE 9

### CD4+ T-cell Responses to HSV Infected DC

Approximately 10⁵ purified CD4⁺ T-cells were derived by stimulation twice biweekly with 5'10⁵ irradiated DC pulsed with recall peptides. The CD4⁺ T-cell effector cells were incubated with X-ray-irradiated DC (T:DC=50:1) that were infected with UV-HSV-1 (3, 1, 0.3. 0.1 multiplicity of infection (MOI)). As control, CD4⁺ T-cells were also incubated with mock infected DC. The DC and CD4⁺ T-cells were incubated for 5 days at 37° C. and (³H)-thymidine was added to the cultures 18 hrs. before harvesting. Proliferative responses were tested in quadruplicated wells, and the results were expressed as mean cpm±SD. In some experiments splenocytes from immunized or control mice were re-stimulated in vitro by incubation with heat-inactivated or UV-inactivated HSV-1.

### EXAMPLE 10

### Infection and In Vivo Depletion of CD4+ and CDB+ T-cells

Mice were infected with 2×10⁵ pfu per eye of HSV-1 in tissue culture media administered as an eye drop in a volume of 10 μl. Beginning 21 days after the second dose of peptide vaccine, some mice were intraperitoneally injected with six doses of 0.1 ml of clarified ascetic fluid in 0.5 ml of PBS containing mAb GK1.5 (anti-CD4) or mAb 2.43 (anti-CD8) on day −7, −1, 0, 2, and 5 post-infection. Flow cytometric analysis of spleen cells consistently revealed a decrease in CD4⁺ and CD8⁺ T-cells in such treated mice to levels of <3% compared to that of normal mice.

### EXAMPLE 11

### Statistical Analysis

Figures represent data from at least two independent experiments. The data are expressed as the mean±SEM and compared by using Student's Hest on a STATVIEW II statistical program (Abacus Concepts, Berkeley, Calif.).

### EXAMPLE 12

### Prediction of gD Epitopes that Elicit Potent CD4⁺ T-cell Responses in Mice with Diverse NHC Backgrounds

The selected peptides were used to immunize H2^{b}, H-2^{d} and H-2^{k} mice and peptide-specific T-cell proliferative responses were determined from spleen and lymph node (LN) cells. Depending on the peptides and strain of mice used, significant proliferative responses were generated by every gD peptide. Thus, each of the twelve chosen regions contained at least one T-cell epitope (FIG. 1). The strongest T-cell responses were directed primarily, although not exclusively, to five peptides (gD₀₋₂₈ (SEQ ID N^{o}11), gD₄₉₋₈₂ (SEQ ID N^{o}2), gD₁₄₆₋₁₇₉ (SEQ ID N^{o}7), gD₂₂₈₋₂₅₇ (SEQ ID N^{o}7), and gD₃₃₂₋₃₅₈ (SEQ ID N^{o}10). The dominant T-cell responses of H-2^{b}, H2^{d} and H-2^{k} mice were focused on the same three peptides (gD₄₉₋₈₂, gD₁₄₆₋₁₇₉, gD₃₃₂₋₃₅₈), suggesting that they contain major T-cell epitopes (FIG. 1). In contrast, gD₂₀₀₋₂₃₄ (SEQ ID N^{o} 4) and gD₂₂₈₋₂₅₇ (SEQ ID N^{o} 8) appeared to be genetically restricted to H2^{d} mice. The levels of response were relatively high with a A cpm >10 000 for most peptides and up to 50,000 cpm for gD₃₃₂₋₃₅₈ (FIG. 1). Although relatively moderate compared to the remaining gD peptides, the responses to gD₂₂₋₅₂ (SEQ ID N^{o}9), gD₇₇₋₁₀₄ (SEQ ID N^{o}6) and gD₉₆₋₁₂₃ (SEQ ID N^{o}5) were also significant (FIG. 1).

The specificity of the proliferative responses was ascertained by the lack of responses after re-stimulation of immune cells with an irrelevant peptide (gB₁₄₁₋₁₆₅) (FIG. 1), and the lack of response to any of the gD peptides in adjuvant-injected control mice (data not shown). FACS analysis of stimulated cells indicated that most responding cells were of CD4⁺ phenotype (FIG. 2). As expected, these responses were blocked by a mAb against CD4⁺ molecules as depicted in Table 2, but not by a mAb against CD8⁺.

TABLE II
CD4+ dependence of T-cell proliferation and cytokine secretion induced by gD peptides ^{(a)}

| | T-cell proliferation (SI) ^{(b, c)} | | | IL-2 (pg/ml)^{©} | | | IFNγ (ng/ml)^{©} | | |
|---|---|---|---|---|---|---|---|---|---|
| Antigen | None | Anti-CD4 | Anti-CD8 | None | Anti-CD4 | Anti-CD8 | None | Anti-CD4 | Anti-CD8 |
| gD₀₋₂₉ | 8 (+/−1) | 1 (+/−1) | 7 (+/−2) | 45 (+/−3) | 12 (+/−2) | 47 (+/−1) | 13 (+/−1) | 5 (+/−3) | 11 (+/−2) |
| gD₄₉₋₈₉ | 13 (+/−2) | 2 (+/−1) | 16 (+/2−) | 92 (+/−5) | 22 (+/−2) | 88 (+/−5) | 60 (+/−4) | 6 (+/−2) | 66 (+/−2) |
| gD₃₃₂₋₃₅₈ | 16 (+/−2) | 3 (+1−2) | 16 (+/1−) | 135 (+/6−) | 36 (+/−1) | 13 (+/−4) | 179 (+/5−) | 4 (+/−1) | 54 (+/−1) |
| UV-HSV | 6 (+/−1) | 3 (+/−2) | 7 (+/−1) | 87 (+/−6) | 16 (+/−1) | 76 (+/−4) | 133 (+/3−) | 4 (+/−1) | 66 (+/−1) |

^{(a)} Splenocytes derived T cells were treated with no Abs (None), or with Abs to CD4 (anti CD4) or CD8 (Anti CD8) molecules and stimulated with the indicated peptides or UV inactivated virus.
^{(b)} The Stimulation Index (SI) was calculated as the mean cpm of cell-associated (3H)-thymidine recovered from wells containing Ag divided by the mean cpm of cell-associated (3H) thymidine recovered from wells without Ag.
^{(c)} Values represent average of data obtained from triplicates (+/−standard deviation)

Collectively, these results showed four new epitope sequences, gD₄₉₋₈₂ (SEQ ID N^{o}2), gD₁₄₆₋₁₇₉ (SEQ ID N^{o}7), gD₂₂₈₋₂₅₇ (SEQ ID N^{o}8) and gD₃₃₂₋₃₅₈ (SEQ ID N^{o}10), that contain major CD4⁺ T-cell sites of gD protein.

### EXAMPLE 13

### Simultaneous Induction of Multiple Ag-specific T-cells to Pools of gD-Derived Peptides

To fully exploit the potential advantages of the peptide-based vaccine approach, the ability of pools of gD peptides to simultaneously induce multiple T-cells specific to each peptide within the pool was explored (FIG. 3). In these experiments, the immunogenicity in H-2^{d} mice of mixed versus individual peptides was compared side by side to investigate if there was any agonistic or synergistic interaction between the peptide sequence bearing at least one epitope composing the pool as a control, H-2^{d} mice were injected with M-ISA-720 alone. Immunization with pool of gD₀₋₂₈, gD₄₉₋₈₂, and gD₃₃₂₋₃₅₈ peptides generated multi-epitopic and significantly higher T-cell responses specific to each peptide (p<0.001) (FIG. 3), Thus, when evaluated individually, each peptide induced a relatively lower response (p<0.001) (FIG. 3). In a similar experiment, the responses induced by a pool of gD₉₆₋₁₂₃ (SEQ ID N^{o}5), gD₁₄₆₋₁₇₉ (SEQ ID N^{o}7)and gD₂₈₇₋₃₁₇ (SEQ ID N^{o}13) peptides were also at a higher level than the responses induced when individual peptides were employed (data not shown).

### EXAMPLE 14

### Determination of Subset of CD4⁺ T-cells Preferentially Induced by Peptides

To determine the type of CD4⁺ T-helper cells involved in lymphocyte proliferation, the inventors studied the pattern of peptide-specific IL-2, IL-4 and IFN-y cytokines induced by each gD peptide. As shown, the gD₀₋₂₈ (SEQ ID N^{o}11), gD₄₉₋₈₂ (SEQ ID N^{o}2), gD₉₆₋₁₂₃ (SEQ ID N^{o}5), gD₁₄₆₋₁₇₉ (SEQ ID N^{o}7), gD₂₂₈₋₂₅₇ (SEQ ID N^{o}8) and gD₃₃₂₋₃₅₉ (SEQ ID N^{o}10) peptides induced Th1 cytokines secretion more efficiently than the remaining peptides (FIG. 4). The gD₂₂₋₅₂ (SEQ ID N^{o}9) and gD₇₇₋₁₀₄ (SEQ ID N^{o}6) peptides preferentially induced Th-2 cytokines. The gD₂₀₀₋₂₃₄ (SEQ ID N^{o}4) peptide induced a mixed response since both IL-4 and IFN-y were induced to a comparable extent (FIG. 4). Overall, for most peptides, the level of IL-2 and IFN-y induced was consistently higher than the level of IL-4, indicating that the selected HSV-1 gD peptides emulsified in the M-ISA-720 adjuvant elicited a polarized Th-1 immune response (FIG. 4). Antibody blocking of T cell activity revealed that cytokines were mainly produced by CD4⁺T-cells and only slightly by CD8⁺ T-cells (Table II).

### EXAMPLE 15

### Determination of Whether T-cells Induced by gD-peptides are Relevant to the Native Viral Protein

To ensure that the observed T-cell responses to the synthetic peptides were reactive to the naturally processed epitopes, the responses to HSV-1 were monitored. T-cells from H-2^{b}, H-2^{d} and H-2^{k} mice immunized with gD₄₉₋₈₂ (SEQ ID N^{o}2), gD₁₄₆₋₁₇₉ (SEQ ID N^{o}7), gD₂₂₈₋₂₅₇ (SEQ ID N^{o}8) and gD₃₃₂₋₃₅₈ (SEQ ID N^{o}10) showed significant proliferation (FIG. 5A) and IFN-y secretion (Table 2) upon in vitro stimulation with UV-inactivated HSV-1. Under the same conditions, T-cells from the adjuvant-injected control mice did not respond to UV-HSV-stimulation (FIG. 5A). Thus, these responses were Ag specific and were not due to a mitogenic effect of viral particles. The HSV-1-specific T cell responses were strongly reduced by anti-CD4⁺ mAb treatment, but not by anti-CD8⁺ mAbs (Table II).

Experiments were performed to determine if the CD4⁺ T-cells induced by gD peptides would recognize the naturally processed viral protein as presented by HSV-1 infected cells. The CD4⁺ T-cell lines specific to gD₀₋₂₈ (SEQ ID N^{o}11), gD₄₉₋₈₂ (SEQ ID N^{o}2), gD₁₄₆₋₁₇₉ (SEQ ID N^{o}7), gD₂₂₈₋₂₅₇ (SEQ ID N^{o}8) or gD₃₃₂₋₃₅₈ (SEQ ID N^{o}10), derived from H-2^{d} mice, responded upon in vitro stimulation with autologous UV-HSV infected bone marrow derived DC (FIG. 5B). No response was observed when mock infected autologous DC were employed as target cells (FIG. 5B). The CD4⁺ T-cells lines induced by gD₇₇₋₁₀₄ (SEQ ID N^{o}6) (FIG. 5B), as well as by gD₂₂₋₅₂ (SEQ ID N^{o}9), gD₁₂₁₋₁₅₂ (SEQ ID N^{o}1), gD₁₇₆₋₂₀₆ (SEQ ID N^{o}3) or gD₂₀₀₋₂₃₄ (SEQ ID N^{o}4) peptides (data not shown) failed to recognize UV-HSV-infected DC. Overall, these results indicated that processing and presentation of the epitopes contained in the gD₀₋₂₈ (SEQ ID N^{o}11), gD₄₉₋₈₂ (SEQ ID N^{o}2), gD₁₄₆₋₁₇₉ (SEQ ID N^{o}7), gD₂₂₈₋₂₅₇ (SEQ ID N^{o}8) and gD₃₃₂₋₃₅₈ (SEQ ID N^{o}10) peptides occurred in HSV infected cells.

### EXAMPLE 16

### Determination of Immunodominance in HSV-primed T-cell Responses to Selected gD-peptides

To define the fine specificity of broadly reactive T-cells associated with viral immunity and to explore immunodominance in the context of HSV infection, proliferation of lymphocytes obtained from twenty HSV-1 infected H-2^{d} mice were evaluated using the twelve gD peptides as Ag (FIG. 6). Although the selected peptides stimulated moderate HSV-specific T-cell responses, surprisingly, the HSV-primed T-cells were reactive to 8 to 10 of the 12 gD peptides, depending on the specific mouse, at the time of analysis. Despite a difference between individual mice, a unique array of T-cell responses was identified for each of the twenty infected mice analyzed. Seven peptides (gD₀₋₂₈ (SEQ ID N^{o}11), gD₄₉₋₈₂ (SEQ ID N^{o}2), gD₉₆₋₁₂₃ (SEQ ID N^{o}5), gD₁₄₆₋₁₇₉ (SEQ ID N^{o}7), gD₂₂₈₋₂₅₇ (SEQ ID N^{o}8), gD₂₈₇₋₃₁₇ (SEQ ID N^{o}13) and gD₃₃₂₋₃₅₈ (SEQ ID N^{o}10)) induced a response in more then 85% of the HSV-infected mice (FIG. 6). The responses were found to gD₀₋₂₈ (SEQ ID N^{o}11), gD₄₉₋₈₂ (SEQ ID N^{o}2), gD₁₄₆₋₁₇₉ (SEQ ID N^{o}7), gD₂₈₇₋₃₁₇ (SEQ ID N^{o}13) and gD₃₃₂₋₃₅₈ (SEQ ID N^{o}10) immunodominant epitopes, and also to gD₂₂₋₅₂ (SEQ ID N^{o}9), gD₇₇₋₁₀₄ (SEQ ID N^{o}6), gD₉₆₋₁₂₃ (SEQ ID N^{o}5), and gD₁₂₁₋₁₅₂ (SEQ ID N^{o}1) that represent subdominant epitopes in H-2^{d} mice. Consistent with their ability to bind l-E^{d} molecule, gD₀₋₂₈ (SEQ ID N^{o}11) and gD₁₄₆₋₁₇₉ (SEQ ID N^{o}7) recalled high T-cell responses in HSV infected H-2^{d} mice (FIG. 6). However, gD₇₇₋₁₀₄ (SEQ ID N^{o}6), gD₂₀₀₋₂₃₄ (SEQ ID N^{o}4) and gD₂₈₇₋₃₁₇ (SEQ ID N^{o}13),that are also strong binders of I-E^{d} molecules, induced either low or no response (FIG. 6). Together these results indicate that the predicted regions contain epitopes that are naturally processed and presented to host's immune system during the course of HSV infection.

### EXAMPLE 17

### Determination of Ability of a Pool of Identified gD-peptide Epitopes to Survive a Lethal HSV-1 Challenge

The gD₄₉₋₈₂ (SEQ ID N^{o}2), gD₁₄₆₋₁₇₉ (SEQ ID N^{o}7), gD₂₂₈₋₂₅₇ (SEQ ID N^{o}8) and gD₃₃₂₋₃₅₈ (SEQ ID N^{o}10) peptides were tested for their ability to provide protective immunity against a lethal challenge with HSV-1 as depicted in Table III. In these experiments, the pools were favored to individual peptides as they elicited higher levels of T-cell responses (FIG. 3). These four peptide epitopes (excluding the previously described protective epitope gD₀₋₂₈) were selected as they were found: i) to generate potent CD4⁺ T-cell responses in mice of diverse MHC background, ii) to elicit the strongest IL-2 and IFN-y production, and iii) to induce T-cells that recognized native viral protein as presented by HSV-1-infected bone marrow derived-dendritic cells, and iv) to recall T-cell response in HSV-1 infected mice.

TABLE III
Immunization with newly identified gD peptides epitopes
in the Montanide's ISA 720 adjuvant confers protective
immunity from a lethal HSV-1 challenge ^{(a)}

| | | | No. | | p versus^{©} |
|---|---|---|---|---|---|
| Mice | | | Protected/ | | gD |
| injected | % of Spleen cells | | No. | % of ^{(b)} | vaccinated |
| with | CD4+ | CD8+ | Tested | Protection | mice |
| gD peptides | 18.1 | 5.6 | 10/10 | 100% | |
| Montanide | 16.3 | 5.1 | 1/10 | 10% | p = 0.0001 |
| None | 15.3 | 4.6 | 1/10 | 10% | p = 0.0001 |

^{(a)} Age and sex matched H-2^{d} mice were immunized with gD₁₄₆₋₁₇₉, gD₂₂₈₋₂₅₇ and gD₃₃₂₋₃₅₈, peptides emulsified in Montanide's ISA 720 adjuvant, injected with Montanide's ISA 720 alone, or left untreated (None). Mice were subsequently challenged with HSV-1 (10⁵ pfu/eye) and monitored daily for lethality.
^{(b)} Results are representative of two independent experiments.
^{(c)} p values comparing the vaccinated mice to the adjuvant injected or non-immunized mice using Student's test

Groups of ten H-2^{d} mice were immunized with a pool of gD₄₉₋₈₂ (SEQ ID N^{o}2), gD₁₄₆₋₁₇₉ (SEQ ID N^{o}7), gD₂₂₈₋₂₅₇ (SEQ ID N^{o}8) and gD₃₃₂₋₃₅₈ (SEQ ID N^{o}10) emulsified in M-ISA-720 adjuvant, injected with M-ISA-720 alone (adjuvant injected control), or left untreated (non-immunized control). Mice were followed for four weeks for their ability to withstand a lethal infection with the McKrae strain of HSV-1. All of the mice that died following challenge did so between day 8 and 12 post-infection. All of the H-2d mice immunized with the pool of gD peptides survived the lethal HSV-1 challenge. In contrast, only 10% of adjuvant-injected and 10% of non-immunized control H-2d mice survived the HSV-1 challenge (Table 3). In a subsequent experiment, H-2^{d} mice immunized with a pool of the weak immunogenic peptides (gD₂₂₋₅₂ (SEQ ID N^{o}9), gD₇₇₋₁₀₄ (SEQ ID N^{o}6), gD₁₂₁₋₁₅₂ (SEQ ID N^{o}1) and gD₂₀₀₋₂₃₄ (SEQ ID N^{o}4)) were comparatively more susceptible to lethal ocular HSV-1 infection (i.e. less then 50% survival). To determine the involvement of CD4⁺ and CD8⁺ T-cells in the induced protection, mice were immunized with gD₄₉₋₈₂ (SEQ ID N^{o}2), gD₁₄₆₋₁₇₉ (SEQ ID N^{o}7), gD₂₂₈₋₂₅₇ (SEQ ID N^{o}8) and gD₃₃₂₋₃₅₈ (SEQ ID N10) peptides and then divided into four groups of ten. The groups were then depleted of CD4⁺ T-cells, depleted of CD8⁺T-cells, left untreated (none), or treated with irrelevant antibodies (rat IgG; IgG control). All four groups were then challenged with HSV-1 as described above. Depletion of CD4⁺T-cells resulted in the death of all infected mice, indicating a significant abrogation of protective immunity as depicted in Table 4. However, depletion of CD8⁺ T-cells or injection of control rat IgG antibodies did not significantly impair the induced protective immunity (p=0,47 and p=1, respectively) (Table IV). These results demonstrate that, in this system, CD4⁺ T-cells are required and CD8⁺ T-cells are not required for protective immunity against lethal HSV-1 challenge.

TABLE IV
Immunization with the newly identified gD peptides epitopes
in the Montanide adjuvant induced a CD4+ T-cell-dependent
protective immunity against a lethal HSV-1 challenge ^{(a)}

| | | | | | p versus^{©} |
|---|---|---|---|---|---|
| | | | No. | | gD |
| Immunized | | | Protected/ | | vaccinated |
| mice treated | % of Spleen cells | | No. | % of ^{(b)} | untreated |
| with | CD4+ | CD8+ | Tested | Protection | mice |
| None | 14.3 | 5.3 | 10/10 | 100% | |
| Anti-CD4 | 0.3 | 4.1 | 0/10 | 0% | p = 0.0001 |
| mAb | | | | | |
| Anti-CD8 | 18.1 | 0.06 | 8/10 | 80% | p = 0.47 |
| mAb | | | | | |
| igG control | 14.7 | 6.7 | 9/10 | 90% | p = 1 |

^{(a)} gD vaccinated H-2^{d} mice were left untreated (None) or depleted of CD4+ or CD8+ T cells by i.p. injections of corresponding mAbs. Control mice received i.p. injections with a rat igG.
^{(b)} Results are representative of two independent experiments.
(c) p values comparing the vaccinated untreated mice to the anti-CD4 mAb, anti-CD8 mAb or IgG treated mice as determined using Student's test.

### EXAMPLE 18

### MHC Class II Binding Assays for the Selection of Promiscuous T Cell Epitopes from gD and gB of HSV-1

### Cell Culture and Purification:

EBV homozygous cell lines PITOUT (DPA1*0103, DPB1*0401), HHKB (DPA1*0103, DPB1*0401), HOM2 (DPA1*0103, DPB1*0401) STEILIN (DRB1*0301, DRB3*0101), and SCHU (DPA1*0103, DPB1*0402) SWEIG (DRB1*1101, DRB3*0202) were used as sources of human HLA-DP and HLA-DR molecules and were from Prof. H. Grosse-Wilde (European Collection for Biomedical Research, Essen, Germany). BOLETH (DRB1*0401, DRB4*0103) and 0206AD (DRB1*1301, DRB3*0101) were kindly provided by Dr. J. Choppin (Hôpital Cochin, Paris) and Prof. J. Dausset (Centre d'Étude du Polymorphisme Humain, Paris), respectively. They were cultured up to 5 10⁹ cells in RPMI medium (Roswell Park Memorial Institute Medium) supplemented by 10% FCS, 2 mM glutamine, 1 mM sodium pyruvate, 500 μg/ml gentamycin, 1% non-essential amino acids (Sigma, St Quentin Fallavier, France). Cells were centrifuged and then lysed on ice at 5×10⁸ cells/ml in 150 mM NaCl, 10 mM Tris-HCl (pH 8.3) buffer containing 1% Nonidet P40, 10 mg/L aprotinin, 5 mM ethylenediaminotetra-acetic acid (EDTA), and 10 □M PMFS (phenylmethylsulfonyl fluoride). After centrifugation at 100,000×g for 1 h, the supernatant was collected. HLA class II molecules were purified by affinity chromatography using the monomorphic mAb L243 for HLA-DR alleles (American Type Culture Collection, Manassas, Va.) or B7/21 for HLA-DP alleles (kind gift from Dr. Y. van de Wal, Department of Immunohematology and Blood Bank, Leiden, The Netherlands). coupled to protein A-Sepharose CL 4B gel (Amersham Pharmacia Biotech, Orsay, France) as described previously by Texier et al. (Texier, C., J. Immunol. 2000, 15;164(6):3177-84). HLA-DR molecules were eluted with 1,1 mM N-dodecyl □-D-maltoside (DM), 500 mM NaCl and 500 mM Na₂CO₃ (pH 11.5).

### HLA-DR and HLA-DP Specific Binding Assays

HLA-DR and HLA-DP molecules were diluted in 10 mM phosphate, 150 mM NaCl, 1 mM DM, 10 mM citrate, and 0.003% thimerosal buffer with an appropriate biotinylated peptide and serial dilutions of competitor peptides. More precisely, HA₃₀₆₋₃₁₈ was used at pH 6 for the DR1 and DR4 and DR51 alleles at 10 nM concentration, and at pH 5 for the DR11 allele at 20 nM concentration. YKL (10 nM) was used for the 701 allele at pH 5 and LOL 191-210 for DR52. Incubation was done at pH 4.5 for the DR15, DR13, and DR3 alleles in the presence of A3₁₅₂₋₁₆₆ (10 nM), B1₂₁₋₃₆ (200 nM), and MT₂₋₁₆ (50 nM), respectively. E2/E168 was used at 10 nM in the presence of DRB4*0101. Oxy₂₇₁₋₂₈₇ at 10 nm were mixed with an appropriate dilution of DP4 molecules (approximately 0.1 μg/ml) and with serial mid-dilutions of competitor peptides. Samples (100 μl per well) were incubated in 96-well polypropylene plates (Nunc, Roskilde, Denmark) at 37° C. for 24 h, except for the DR13, DR3 and DR53 alleles which were incubated 72 h, neutralized and applied to B7/21(for DP4 alleles) or L243 (for DR alleles) coated plates for 2 h. Bound biotinylated peptide was detected by means of streptavidin-alkaline phosphatase conjugate (Amersham, Little Chalfont, U.K.), and 4-methylumbelliferyl phosphate substrate (Sigma, St Quentin Fallavier, France). Emitted fluorescence was measured at 450 nm upon excitation at 365 nm in a Victor II spectrofluorimeter (Perkin Elmer Instruments, Les Ulis, France). Data were expressed as the peptide concentration that prevented binding of 50% of the labeled peptide (IC₅₀). Validity of each experiments was assessed by reference peptides.

NT=not tested.

### List of HLA-DR and HLA-DP Molecules and Biotinylated Tracers Used in This Study.

| | | | | |
|---|---|---|---|---|
| | | Frequen- | | |
| specific- | | cies | | IC50 |
| ities | alleles | (%) | Tracer | (nM) |

| | | | | | |
|---|---|---|---|---|---|
| DR1 | DR (α1*0101, α*0 | 9, 3 | HA (307- | PKYVKQNTLKLAT | 2 |
| | 101) | | 319) | | |
| DR3 | DR (α1*0101, α1*0 | 10, 9 | MT (2-16) | AKTIAYDEEARRGLE | 305 |
| | 301) | | | | |
| DR4 | DR (α1*0101, α1*0 | 5, 6 | HA (307- | PKYVKQNTLKLAT | 42 |
| | 401) | | 319) | | |
| DR7 | DR (α1*0101, α1*0 | 14 | YKL | AAYAAAKAAALAA | 6 |
| | 701) | | | | |
| DR11 | DR (α1*0101, α1*1 | 9, 2 | HA (307- | PKYVKQNTLKLAT | 52 |
| | 101) | | 319) | | |
| DR13 | DR (α1*0101, α1*1 | 6 | B1 (21-36) | TERVRLVTRHIYNREE | 276 |
| | 301) | | | | |
| DR15 | DR (α1*0101, α1*1 | 8 | A3 (152- | EAEQLRAYLDGTGVE | 13 |
| | 501) | | 166) | | |
| DR51 | DR (α1*0101α5*01 | 15 | HA (307- | PKYVKQNTLKLAT | 12 |
| | 01) | | 319) | | |
| DR52 | DR (α1*0101, α3*0 | 18 | LOL (191- | ESWGAVWRIDTPDKLT | 15 |
| | 101) | | 210) | GPFT | |
| DR53 | DR (α1*0101, α4*0 | 49 | E2/E168 | ESWGAVWRIDTPDKLT | 16 |
| | 101) | | | GPFT | |
| DP401 | DP (α1*0101, α1*0 | 64 | bOxy 271- | EKKYFAATQFEPLAAR | 10 |
| | 401) | | 287 | | |
| DP402 | DP (α1*0101, α1*0 | 21 | bOxy 271- | EKKYFAATQFEPLAAR | 7 |
| | 402) | | 287 | | |

The phenotypic frequencies are from the French population and are representative of other Caucasian populations (from HLA: Fonctions immunitaires et applications médicales. Colombani J., John Libbey. Eurotext). The IC50 values are obtained in the preliminary experiments and serve as references in the following experiments.

The results of HLA class II binding assays are presented in Table V and VI. Data were expressed as the peptide concentration that prevented binding of 50% of the labeled peptide (IC50). Average and SE values were deduced from at least three independent experiments. Validity of each experiments was assessed by reference peptides.

While the description above refers to particular embodiments of the present invention, it will be understood that many modifications may be made without departing from the spirit thereof. For instance, the peptides of the present invention may be used in the treatment of any number of variations of HSV where observed, as would be readily recognized by one skilled in the art and without undue experimentation. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

TABLE V
Threshold 1000 nM/3 alleles

Class II MHC alleles

| | | | | | | | | | | |
|---|---|---|---|---|---|---|---|---|---|---|
| Name | Source | position | Sequence | DR1 | DR3 | DR4 | DR7 | DR11 | DR13 | DR15 |

| | | | | | | | | | | |
|---|---|---|---|---|---|---|---|---|---|---|
| HSV | gD | 121-152 | NKSLGACPIRTQPRWNYYDSFSAVSEDNLGFL | 53 | 66 | 8 | 19 | 289 | 160 | 2 |
| 33 | | | | | | | | | | |
| HSV | gB | 809-840 | KLAEAREMIRYMALVSAMERTEHKAKKKOTSA | 6 | 995 | 37 | 296 | 13 | 284 | 3 |
| 1 | | | | | | | | | | |
| HSV | gB | 765-799 | FRYVMRLQSNPMKALYPLTTKELKNFTNPDASGEG | 2 | 4775 | 12 | 20 | 4 | 314 | 3 |
| 8 | | | | | | | | | | |
| HSV | gB | 401-433 | ATHIKVGQPQYYLANGGFLIAYQFLLSNTLAEL | <1 | >100000 | 33 | 1 | 72 | >100000 | 60 |
| 2 | | | | | | | | | | |
| HSV | gB | 111-140 | NYTEGIAVVFKLENIAPYKFKATMYYKDVTV | 343 | 1271 | 29 | 56 | 170 | 500 | 30 |
| 3 | | | | | | | | | | |
| HSV | gB | 243-282 | VEEVDARSVYPYDEFVLAGDFVYMSPFYGYREGSHTEHT | 1 | 4000 | 37 | 64 | 61 | 35355 | 1 |
| 6 | | | | | | | | | | |
| HSV | gB | 631-661 | RADITTTVSTFIDLNTMLEDHEFVPLEVYTR | 27 | >100000 | 524 | 1500 | 110 | >100000 | 60 |
| 7 | | | | | | | | | | |
| HSV | gB | 453-483 | PPGASANASVERIKTTSSIEFARLQFTYNHI | 178 | >100000 | 705 | 30 | 432 | >100000 | 264 |
| 11 | | | | | | | | | | |
| HSV | gD | 146-179 | EDNLGFLMHAPAFETAGTYLRLVKINDWTEITQF | 40 | 10247 | 632 | 316 | 175 | >100000 | 35 |
| 34 | | | | | | | | | | |
| HSV | gD | 49-82 | QPPSLPITVYYAVLERACRSVLLNAPSEAPQIVR | 3 | 1249 | 93 | 173 | 120 | >100000 | 18 |
| 36 | | | | | | | | | | |
| HSV | gD | 200-234 | SACLSPQAYQQGVTVDSIGMLPRFIFENQRTVAVY | 4 | 307 | 40 | 200 | 44 | 2049 | 13 |
| 37 | | | | | | | | | | |
| HSV | gD | 176-206 | TQFILEHRAKGSCKYALPLRRIPSACLSPQ | 54 | 1342 | 955 | 21 | 5 | 200 | 76 |
| 38 | | | | | | | | | | |
| HSV | gB | 424-445 | PLLSNTLAELYVREHLREQSRK | 30 | >100000 | 1778 | 95 | 612 | 539 | 163 |
| 4 | | | | | | | | | | |
| HSV | gB | 590-812 | NNELRLTRDAIEPCTVGHRRYFT | 412 | 164 | 59 | 42 | 1876 | 2612 | 751 |
| 13 | | | | | | | | | | |
| HSV | gB | 607-634 | HRRYFTFGGGYVYFEEYAYSHQLSRADT | 45 | >100000 | 5593 | 150 | 367 | 1225 | 169 |
| 14 | | | | | | | | | | |
| HSV | gD | 96-123 | TIAWFRMGGNCAIPITVMEYTECSYNKS | 3 | NT | 61 | 37 | 598 | 4762 | 167 |
| 41 | | | | | | | | | | |
| HSV | gD | 0-28 | SKYALVDASLKMADPNRFRGKDLPVLDQL | 56 | 78 | 58 | 374 | 648 | >100000 | 10954 |
| 30 | | | | | | | | | | |
| HSV | gD | 22-52 | DLPVLDQLTDPPGVRRVVHIQAGLFDPFQPPS | 3 | 2492 | 63 | 224 | 25 | >100000 | 787 |
| 31 | | | | | | | | | | |
| HSV | gD | 332-358 | ICGIVYWMRRHTQKAPKRIRL | 150 | 1643 | 5872 | 274 | 5 | 56 | 950 |
| 39 | | | | | | | | | | |
| HSV | gB | 80-106 | DANFYVCPPPTGATVVQFEQPRRCPTR | 74 | 9539 | 366 | 725 | 529 | 2298 | 669 |
| 10 | | | | | | | | | | |
| HSV | gD | 77-104 | APQIVRGASEDVRKQPYNLTIAWFRMGG | 22 | 2349 | NT | 4 | 300 | NT | 25 |
| 32 | | | | | | | | | | |
| HSV | gB | 173-204 | AKGVCRSTAKYVRNNLETTAFHRDDHETDMEL | 262 | 2045 | 3969 | 141 | 1225 | 2450 | 3779 |
| 5 | | | | | | | | | | |
| HSV | gB | 837-870 | GTSALLSAKVTDMVMRKRRNTNYTQVPNKDGDAD | 493 | 11402 | 4000 | 229 | 424 | 362 | 2432 |
| 9 | | | | | | | | | | |
| HSV | gB | 568-594 | SRPLVSFRYEDQGPLVEGQLGENNELR | 15 | >100000 | 659 | 794 | 5138 | >100000 | 88 |
| 12 | | | | | | | | | | |
| HSV | gD | 1-23 | KYALVDASLKMADFNRFRGKDLP | 1225 | 120 | 82 | 894 | 5254 | >100000 | 24495 |
| 29 | | | | | | | | | | |
| HSV | gD | 228-257 | QRTVAVYSLKIAGWHGPKAFYTSTLLFFEL | 1162 | 2392 | 9920 | 20 | 39 | 1587 | 2 |
| 40 | | | | | | | | | | |
| HSV | gD | 287-317 | APQIPPNWHIPSIQDAATPYHPPATPNNMGL | 3162 | 19494 | 600 | 2449 | 25000 | >100000 | 6788 |
| 35 | | | | | | | | | | |

Class II MHC alleles

| | | | | | | | | | |
|---|---|---|---|---|---|---|---|---|---|
| Name | Source | position | Sequence | DRB3 | DRB4 | DRB5 | DP401 | DP402 | Range |

| | | | | | | | | | |
|---|---|---|---|---|---|---|---|---|---|
| HSV | gD | 121-152 | NKSLGACPIRTQPRWNYYDSFSAVSEDNLGFL | 226 | 319 | 134 | 83 | 65 | 12 |
| 33 | | | | | | | | | |
| HSV | gB | 809-840 | KLAEAREMIRYMALVSAMERTEHKAKKKOTSA | >100000 | 43 | 6 | 1612 | 240 | 10 |
| 1 | | | | | | | | | |
| HSV | gB | 765-799 | FRYVMRLQSNPMKALYPLTTKELKNFTNPDASGEG | 55000 | 232 | 2 | 107 | 32 | 10 |
| 8 | | | | | | | | | |
| HSV | gB | 401-433 | ATHIKVGQPQYYLANGGFLIAYQFLLSNTLAEL | >100000 | 787 | 160 | 32 | 34 | 9 |
| 2 | | | | | | | | | |
| HSV | gB | 111-140 | NYTEGIAVVFKLENIAPYKFKATMYYKDVTV | 1597 | 2510 | 25 | 80 | 45 | 9 |
| 3 | | | | | | | | | |
| HSV | gB | 243-282 | VEEVDARSVYPYDEFVLAGDFVYMSPFYGYREGSHTEHT | 102 | NT | 9 | 102 | 16 | 9 |
| 6 | | | | | | | | | |
| HSV | gB | 631-661 | RADITTTVSTFIDLNTMLEDHEFVPLEVYTR | 663 | 401 | 58 | 155 | 76 | 9 |
| 7 | | | | | | | | | |
| HSV | gB | 453-483 | PPGASANASVERIKTTSSIEFARLQFTYNHI | >100000 | 498 | 406 | 424 | 57 | 9 |
| 11 | | | | | | | | | |
| HSV | gD | 146-179 | EDNLGFLMHAPAFETAGTYLRLVKINDWTEITQF | 2020 | 743 | 85 | 115 | 164 | 9 |
| 34 | | | | | | | | | |
| HSV | gD | 49-82 | QPPSLPITVYYAVLERACRSVLLNAPSEAPQIVR | 5000 | 170 | 66 | 615 | 98 | 9 |
| 36 | | | | | | | | | |
| HSV | gD | 200-234 | SACLSPQAYQQGVTVDSIGMLPRFIFENQRTVAVY | 41 | 3742 | 68 | 1597 | 167 | 9 |
| 37 | | | | | | | | | |
| HSV | gD | 176-206 | TQFILEHRAKGSCKYALPLRRIPSACLSPQ | 25000 | 1803 | 91 | 91 | 145 | 9 |
| 38 | | | | | | | | | |
| HSV | gB | 424-445 | PLLSNTLAELYVREHLREQSRK | >100000 | 15000 | 671 | 890 | 240 | 8 |
| 4 | | | | | | | | | |
| HSV | gB | 590-812 | NNELRLTRDAIEPCTVGHRRYFT | 677 | 240 | 55 | >100000 | >100000 | 8 |
| 13 | | | | | | | | | |
| HSV | gB | 607-634 | HRRYFTFGGGYVYFEEYAYSHQLSRADT | >100000 | 310 | 22 | 145 | 81 | 8 |
| 14 | | | | | | | | | |
| HSV | gD | 96-123 | TIAWFRMGGNCAIPITVMEYTECSYNKS | >100000 | 1672 | 102 | 257 | 88 | 8 |
| 41 | | | | | | | | | |
| HSV | gD | 0-28 | SKYALVDASLKMADPNRFRGKDLPVLDQL | 535 | >100000 | 7 | 17889 | 3795 | 7 |
| 30 | | | | | | | | | |
| HSV | gD | 22-52 | DLPVLDQLTDPPGVRRVVHIQAGLFDPFQPPS | 5979 | 397 | 58 | 62032 | 46990 | 7 |
| 31 | | | | | | | | | |
| HSV | gD | 332-358 | ICGIVYWMRRHTQKAPKRIRL | 2307 | 703 | 31 | NT | >100000 | 7 |
| 39 | | | | | | | | | |
| HSV | gB | 80-106 | DANFYVCPPPTGATVVQFEQPRRCPTR | >100000 | 7416 | 520 | NT | 6841 | 6 |
| 10 | | | | | | | | | |
| HSV | gD | 77-104 | APQIVRGASEDVRKQPYNLTIAWFRMGG | >100000 | NT | 1 | 1449 | 381 | 6 |
| 32 | | | | | | | | | |
| HSV | gB | 173-204 | AKGVCRSTAKYVRNNLETTAFHRDDHETDMEL | 224 | 90000 | 675 | 1549 | 547 | 5 |
| 5 | | | | | | | | | |
| HSV | gB | 837-870 | GTSALLSAKVTDMVMRKRRNTNYTQVPNKDGDAD | 58000 | 16000 | 559 | 8000 | 4000 | 5 |
| 9 | | | | | | | | | |
| HSV | gB | 568-594 | SRPLVSFRYEDQGPLVEGQLGENNELR | 290 | 1643 | 1549 | 1949 | 1775 | 5 |
| 12 | | | | | | | | | |
| HSV | gD | 1-23 | KYALVDASLKMADFNRFRGKDLP | 1396 | 52536 | 8 | 17550 | 1629 | 4 |
| 29 | | | | | | | | | |
| HSV | gD | 228-257 | QRTVAVYSLKIAGWHGPKAFYTSTLLFFEL | >100000 | 1163 | 22 | 1361 | 7211 | 4 |
| 40 | | | | | | | | | |
| HSV | gD | 287-317 | APQIPPNWHIPSIQDAATPYHPPATPNNMGL | 5000 | 3256 | 4500 | >100000 | >100000 | 1 |
| 35 | | | | | | | | | |

TABLE VI
Threshold 600 nM/5 alleles

Class II MHC alleles

| | | Posi- | | | | | | | | |
|---|---|---|---|---|---|---|---|---|---|---|
| Name | Source | tion | Sequence | DR1 | DR3 | DR4 | DR7 | DR11 | DR13 | DR15 |
| HSV | gD | 121- | NKSLGACPIRTQPRWNYYDSFSAVSEIRNLGFL | 53 | 66 | 6 | 19 | 289 | 160 | 2 |
| 33 | | 152 | | | | | | | | |
| HSV | gB | 809- | KLAEAREMIRYMALVSAMERTEHKAKKKOTSA | 6 | 995 | 37 | 296 | 13 | 284 | 3 |
| 1 | | 840 | | | | | | | | |
| HSV | gB | 765- | FRYVMRLQSNPMKALYPLTTKELKNPDASGEG | 2 | 4775 | 12 | 20 | 4 | 314 | 3 |
| 8 | | 799 | | | | | | | | |
| HSV | gB | 401- | ATHIKVOQPQYYLANOGFLIAYPLLSNTLAEL | <1 | >100000 | 33 | 1 | 72 | >100000 | 60 |
| 2 | | 433 | | | | | | | | |
| HSV | gB | 111- | NYTEGIAVVFKENIAPYKFKATMYYKDVTV | 343 | 1271 | 29 | 56 | 170 | 500 | 30 |
| 3 | | 140 | | | | | | | | |
| HSV | gB | 243- | VEEVDARSVYPYDEFVLATGDFVYMSPFYGYREQSHTEHT | 1 | 4000 | 37 | 64 | 61 | 35355 | 1 |
| 6 | | 282 | | | | | | | | |
| HSV | gB | 631- | RADITTVSTFIDLNITMLEDHEFVPLEVYTR | 27 | >100000 | 524 | 1500 | 110 | >100000 | 60 |
| 7 | | 661 | | | | | | | | |
| HSV | gB | 453- | PPGASANASVERIKTTSSIEFARLQFTYNHI | 178 | >100000 | 705 | 30 | 432 | >100000 | 264 |
| 11 | | 483 | | | | | | | | |
| HSV | gD | 146- | EDNLGFLMHAPAFETAGTYLRLVKINDWTEITQF | 40 | 10247 | 632 | 316 | 175 | >100000 | 35 |
| 34 | | 179 | | | | | | | | |
| HSV | gD | 49- | QPPSLPITVYVAVLERACRSVLLNAPSEAPQIVR | 3 | 1249 | 93 | 173 | 120 | >100000 | 18 |
| 36 | | 82 | | | | | | | | |
| HSV | gD | 200- | SACLSPQAYQQGVTVDSIOMLPRFIPENQRTVAVY | 4 | 307 | 40 | 200 | 44 | 2049 | 13 |
| 37 | | 234 | | | | | | | | |
| HSV | gD | 176- | TQFILEHRAKOSCKYALPLRIPPSACLSPQ | 54 | 1342 | 955 | 21 | 5 | 200 | 76 |
| 38 | | 206 | | | | | | | | |
| HSV | gB | 590- | NNELRLTRDAIEPCTVGHRRYFT | 412 | 164 | 59 | 42 | 1876 | 2612 | 751 |
| 13 | | 612 | | | | | | | | |
| HSV | gB | 607- | HRRYFTFGGGYVYFEEYAYSHQLSRADI | 45 | >100000 | 5593 | 150 | 387 | 1225 | 169 |
| 14 | | 634 | | | | | | | | |
| HSV | gD | 96- | TIAWFRMGGNCAIPITVMEYTECSYNKS | 3 | NT | 61 | 37 | 598 | 4762 | 167 |
| 41 | | 123 | | | | | | | | |
| HSV | gB | 424- | FLLSNTLAELYVREHLREQSRK | 30 | >100000 | 1778 | 95 | 612 | 539 | 163 |
| 4 | | 445 | | | | | | | | |
| HSV | gD | 0- | SKYALVDASLKMADPNRFRGKDLPVLDQL | 58 | 79 | 58 | 374 | 648 | >100000 | 10954 |
| 30 | | 28 | | | | | | | | |
| HSV | gD | 22- | DLPVLDQLTDPPGVRRVYHIQAGLPDPFQPPS | 3 | 2492 | 63 | 224 | 25 | >100000 | 787 |
| 31 | | 52 | | | | | | | | |
| HSV | gD | 332- | ICGIVYWMRIHTQKAPKRIRL | 150 | 1643 | 5872 | 274 | 5 | 56 | 950 |
| 39 | | 358 | | | | | | | | |
| HSV | gB | 80- | DANFYVCPPPTGATVVQFEQPRRCPTR | 74 | 9539 | 366 | 725 | 529 | 2298 | 669 |
| 10 | | 106 | | | | | | | | |
| HSV | gD | 77- | APQIVRGASEDVRKQPYNLTIAWFRMGG | 22 | 2349 | NT | 4 | 300 | NT | 25 |
| 32 | | 104 | | | | | | | | |
| HSV | gB | 173- | AKGVCRSTAKYVRNNLETTAFHRDDHETDMEL | 262 | 2045 | 3969 | 141 | 1225 | 2450 | 3779 |
| 5 | | 204 | | | | | | | | |
| HSV | gB | 837- | GTSALLSAKVTDMVMRKRRNTNYTQVPNKDGDAD | 493 | 11402 | 4000 | 229 | 424 | 362 | 2432 |
| 9 | | 870 | | | | | | | | |
| HSV | gB | 568- | SRPLVSFRYEDQGFLVEGQLGENNELR | 15 | >100000 | 659 | 794 | 5138 | >100000 | 88 |
| 12 | | 594 | | | | | | | | |
| HSV | gD | 228- | QRTVAVYSLRIAGWHGPKAPYTSTLLFFEL | 1162 | 2392 | 9920 | 20 | 39 | 1587 | 2 |
| 40 | | 257 | | | | | | | | |
| HSV | gD | 1- | KYALVDASLKMADPNRFRKGKDLP | 1225 | 120 | 82 | 894 | 5254 | >100000 | 24495 |
| 29 | | 23 | | | | | | | | |
| HSV | gD | 287- | APQIPPNWHIPSIQDAATPVHPPATPNNMGL | 3162 | 19494 | 600 | 2449 | 25000 | >100000 | 6788 |
| 35 | | 317 | | | | | | | | |

Class II MHC alleles

| | | Posi- | | | | | | | |
|---|---|---|---|---|---|---|---|---|---|
| Name | Source | tion | Sequence | DRD3 | DRB4 | DRB5 | DP401 | DP402 | Range |
| HSV | gD | 121- | NKSLGACPIRTQPRWNYYDSFSAVSEIRNLGFL | 226 | 319 | 134 | 83 | 65 | 12 |
| 33 | | 152 | | | | | | | |
| HSV | gB | 809- | KLAEAREMIRYMALVSAMERTEHKAKKKOTSA | >100000 | 43 | 6 | 1612 | 240 | 10 |
| 1 | | 840 | | | | | | | |
| HSV | gB | 765- | FRYVMRLQSNPMKALYPLTTKELKNPDASGEG | 55000 | 232 | 2 | 107 | 32 | 10 |
| 8 | | 799 | | | | | | | |
| HSV | gB | 401- | ATHIKVOQPQYYLANOGFLIAYPLLSNTLAEL | >100000 | 787 | 160 | 32 | 34 | 9 |
| 2 | | 433 | | | | | | | |
| HSV | gB | 111- | NYTEGIAVVFKENIAPYKFKATMYYKDVTV | 1597 | 2510 | 25 | 60 | 45 | 9 |
| 3 | | 140 | | | | | | | |
| HSV | gB | 243- | VEEVDARSVYPYDEFVLATGDFVYMSPFYGYREQSHTEH | 102 | NT | 9 | 102 | 16 | 9 |
| 6 | | 282 | | | | | | | |
| HSV | gB | 631- | RADITTVSTFIDLNITMLEDHEFVPLEVYTR | 663 | 401 | 58 | 155 | 78 | 9 |
| 7 | | 661 | | | | | | | |
| HSV | gB | 453- | PPGASANASVERIKTTSSIEFARLQFTYNHI | >100000 | 498 | 406 | 424 | 57 | 9 |
| 11 | | 483 | | | | | | | |
| HSV | gD | 146- | EDNLGFLMHAPAFETAGTYLRLVKINDWTEITQF | 2020 | 743 | 85 | 115 | 164 | 9 |
| 34 | | 179 | | | | | | | |
| HSV | gD | 49- | QPPSLPITVYVAVLERACRSVLLNAPSEAPQIVR | 5000 | 170 | 66 | 615 | 98 | 9 |
| 36 | | 82 | | | | | | | |
| HSV | gD | 200- | SACLSPQAYQQGVTVDSIOMLPRFIPENQRTVAVY | 41 | 3742 | 68 | 1597 | 167 | 9 |
| 37 | | 234 | | | | | | | |
| HSV | gD | 176- | TQFILEHRAKOSCKYALPLRIPPSACLSPQ | 25000 | 1803 | 91 | 91 | 145 | 8 |
| 38 | | 206 | | | | | | | |
| HSV | gB | 590- | NNELRLTRDAIEPCTVGHRRYFT | 677 | 240 | 55 | >100000 | >100000 | 8 |
| 13 | | 612 | | | | | | | |
| HSV | gB | 607- | HRRYFTFGGGYVYFEEYAYSHQLSRADI | >100000 | 310 | 22 | 145 | 81 | 8 |
| 14 | | 634 | | | | | | | |
| HSV | gD | 96- | TIAWFRMGGNCAIPITVMEYTECSYNKS | >100000 | 1672 | 102 | 267 | 88 | 8 |
| 41 | | 123 | | | | | | | |
| HSV | gB | 424- | FLLSNTLAELYVREHLREQSRK | >100000 | 15000 | 671 | 890 | 240 | 7 |
| 4 | | 445 | | | | | | | |
| HSV | gD | 0- | SKYALVDASLKMADPNRFRGKDLPVLDQL | 535 | >100000 | 7 | 17689 | 3795 | 7 |
| 30 | | 28 | | | | | | | |
| HSV | gD | 22- | DLPVLDQLTDPPGVRRVYHIQAGLPDPFQPPS | 6979 | 397 | 58 | 62032 | 48990 | 7 |
| 31 | | 52 | | | | | | | |
| HSV | gD | 332- | ICGIVYWMRIHTQKAPKRIRL | 2307 | 703 | 31 | NT | >100000 | 6 |
| 39 | | 358 | | | | | | | |
| HSV | gB | 80- | DANFYVCPPPTGATVVQFEQPRRCPTR | >100000 | 7416 | 520 | NT | 6841 | 6 |
| 10 | | 106 | | | | | | | |
| HSV | gD | 77- | APQIVRGASEDVRKQPYNLTIAWFRMGG | >100000 | NT | 1 | 1449 | 381 | 6 |
| 32 | | 104 | | | | | | | |
| HSV | gB | 173- | AKGVCRSTAKYVRNNLETTAFHRDDHETDMEL | 224 | 90000 | 675 | 1549 | 547 | 5 |
| 5 | | 204 | | | | | | | |
| HSV | gB | 837- | GTSALLSAKVTDMVMRKRRNTNYTQVPNKDGDAD | 58000 | 16000 | 559 | 8000 | 4000 | 5 |
| 9 | | 870 | | | | | | | |
| HSV | gB | 568- | SRPLVSFRYEDQGFLVEGQLGENNELR | 290 | 1643 | 1549 | 1949 | 1775 | 5 |
| 12 | | 594 | | | | | | | |
| HSV | gD | 228- | QRTVAVYSLRIAGWHGPKAPYTSTLLFFEL | >100000 | 1163 | 22 | 1361 | 7211 | 4 |
| 40 | | 257 | | | | | | | |
| HSV | gD | 1- | KYALVDASLKMADPNRFRKGKDLP | 1396 | 52536 | 8 | 17550 | 1629 | 3 |
| 29 | | 23 | | | | | | | |
| HSV | gD | 287- | APQIPPNWHIPSIQDAATPVHPPATPNNMGL | 5000 | 3256 | 4500 | >100000 | >100000 | 1 |
| 35 | | 317 | | | | | | | |

1) Immunogenic composition comprising at least one Herpes Simplex Virus type 1 (HSV-1) and/or type 2 (HSV-2) epitope containing peptide from glycoprotein D (gD) and/or glycoprotein B (gB), a pharmaceutical carrier and/or a human compatible adjuvant, wherein said epitope containing peptide having the capacity to bind on at least three alleles of humans HLA class II molecules having a frequency superior to 5% in a Caucasian population, with a binding activity less or equal to 1000 nanomolar.

2) Immunogenic composition according to claim 1, wherein said epitope containing peptide having the capacity to bind on at least five alleles of humans HLA class II molecules having a frequency superior to 5% in a Caucasian population, with a binding activity less or equal to 800 nanomolar.

3) Immunogenic composition according to claim 1, wherein said epitope containing peptide is selected from the group of peptide sequences consisting of SEQ ID N^{o}1 to SEQ ID N^{o}12, SEQ ID N^{o}14 to SEQ ID N^{o}25, SEQ ID N^{o}28 to SEQ ID N^{o}39, and SEQ ID N^{o}41 to SEQ ID N^{o}52, or fragments thereof.

4) Immunogenic composition according to claims 1 to 3, wherein it comprises a combination of 2 to 8 epitope containing peptides.

5) Immunogenic composition according to claim 4, wherein it comprises a combination of 3 to 7 epitope containing peptides from gD HSV-1 selected from the group of peptide sequences consisting of SEQ ID N^{o}2, SEQ ID N^{o}5, SEQ ID N^{o}7, SEQ ID N^{o}8, SEQ ID N^{o}10, SEQ ID N^{o}11 and SEQ ID N^{o}12, preferably a combination of 3 to 5 epitope containing peptides selected from the group of peptide sequences consisting of SEQ ID N^{o}2, SEQ ID N^{o}7, SEQ ID N^{o}8, SEQ ID N^{o}10, and SEQ ID N^{o}11, and more preferably a combination of 4 epitope containing peptide selected from the group of peptide sequences consisting of SEQ ID N^{o}2, SEQ ID N^{o}7, SEQ ID N^{o}8 and SEQ ID N^{o}10, and/or the corresponding gD HSV-2 epitope containing peptides, or combinations of said gD HSV-1 and gD HSV-2 epitope containing peptides.

6) Immunogenic composition according to claim 5, wherein the corresponding HSV-2 epitope containing peptides present an homology of the peptide sequence with the HSV-1 epitope containing peptide of at least 70%, preferably at least 80%, more preferably at least 90%.

7) Immunogenic composition according to claim 1, wherein the epitope containing peptide is in an amount from about 50 μg to about 5 mg.

8) Immunogenic composition according to claim 1, wherein the human compatible adjuvant is the Montanide ISA 720, in an amount from about 15 μl to about 25 μl.

9) Immunogenic composition according to claim 1, wherein the pharmaceutical carrier is selected from the group consisting of water, alcohol, natural or hardened oil, natural or hardened wax, calcium carbonate, sodium carbonate, calcium phosphate, kaolin, talc, lactose, lipid tail and combination thereof, in an amount of about 10 μl to about 100 μl.

10) Immunogenic composition according to claim 1, further comprising an additional component selected from the group consisting of a vehicle, an additive, an excipient, a pharmaceutical adjunct, a therapeutic compound or agent useful in the treatment of HSV and combinations thereof.

11) Immunogenic composition according to claim 1, wherein the composition is formulated to be administered by a technique selected from the group consisting of systemic injection, mucosal administration, topical administration, spray, drop, aerosol, gel and sweet formulation, and particularly is formulated to be administered by systemic injection, more particularly by subcutaneous injection.

12) Immunogenic composition according to claim 1 for use as a medicament.

13) Use of an immunogenic composition according to claim 1 for the manufacture of a medicament for prevention or treatment of a condition selected from the group consisting of HSV-1 primary infections, HSV-1 recurrences, HSV-2 primary infection, HSV-2 recurrences, cold sores, genital lesions, corneal blindness, and encephalitis, a condition in which a stimulation of IL-2 and IFN-γ is desirable and in which the induction of the Th-1 subset of T-cells is desirable.

14) HSV-1 or HSV-2 peptide sequence bearing at least one epitope, or fragment thereof, wherein said peptide sequence is selected from the group consisting of SEQ ID N^{o}1 to SEQ ID N^{o}11, SEQ ID N^{o}14 to SEQ ID N^{o}52.

15) Use of peptide sequence according to claim 14 for the manufacture of a medicament for treating or preventing a condition related to HSV-1 and/or HSV-2, and of a diagnosis reagent.
